(19) United States
(12) Patent Application Publication
(10) Pub. No.: US 2004/0225006 A1
(43) Pub. Date: Nov. 11, 2004

(54) (Hetero) bicyclymethanesulfonylamino-substituted hydroxamic acid derivates

(75) Inventors: Gordon Bruton, Sawbridgeworth (GB); Andrew Faller, Epping (GB); Barry Sidney Orlek, Epping (GB); Kishore Kalidas Rana, Welwyn Garden City (GB); Graham Walker, Reigate (GB)

Correspondence Address: GLAXOSMITHKLINE, Corporate Intellectual Property - UW2220, P.O. Box 1539, King of Prussia, PA, 19406-0939, US

(73) Assignee: SmithKline Beecham p.l.c.

(21) Appl. No.: 10/846,286

(22) Filed: May 14, 2004

Related U.S. Application Data
(63) Continuation of application No. 10/168,461, filed on Mar. 24, 2003, now abandoned.
Application No. 10/168,461 is a 371 of international application No. PCT/GB00/04941, filed on Dec. 21, 2000.

(30) Foreign Application Priority Data
Dec. 24, 1999 (GB) 9930687.0
Nov. 1, 2000 (GB) 0026693.2

(51) Int. Cl. A61K 31/38; A61K 31/19; C07C 311/47

(52) U.S. Cl. 514/438; 514/443; 514/575; 549/55; 562/623

(57) ABSTRACT

Compounds of formula (I) wherein R is hydrogen, alkyl, alkenyl, alkynyl, aryl, heteroaryl or heterocyclyl; and R¹ is bicyclyl or heterobicyclyl; are useful in the treatment and prophylaxis of conditions mediated by s-CD23 or TNF.

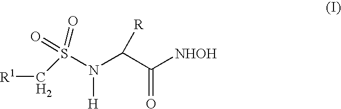

This invention relates to novel inhibitors of the formation of soluble human CD23 and their use in the treatment of conditions associated with excess production of soluble CD23 (s-CD23) such as autoimmune disease, inflammation and allergy.

CD23 (the low affinity IgE receptor FceRII, Blast 2), is a 45 kDa type II integral protein expressed on the surface of a variety of mature cells, including B and T lymphocytes, macrophages, natural killer cells, Langerhans cells, monocytes and platelets (Delespesse et al, Adv Immunol, 49 [1991] 149-191). There is also a CD23-like molecule on eosinophils (Grangette et al, J Immunol, 143 [1989] 3580.3588). CD23 has been implicated in the regulation of the immune response (Delespesse et al, Immunol Rev, 125 [1992] 77-97). Human CD23 exists as two differentially regulated isoforms, a and b, which differ only in the amino acids at the intracellular N-terminus (Yokota et al, Cell, 55 [1988] 611-618). In man the constitutive a isoform is found only on B-lymphocytes, whereas type b, inducible by IL4, is found on all cells capable of expressing CD23.

Intact, cell bound CD23 (i-CD23) is known to undergo cleavage from the cell surface leading to the formation of a number of well-defined soluble fragments (s-CD23), which are produced as a result of a complex sequence of proteolytic events, the mechanism of which is still poorly understood (Bourget et al J Biol Chem, 269 (1994) 6927-6930). Although not yet proven, it is postulated that the major soluble fragments (Mr 37, 33, 29 and 25 kDa) of these proteolytic events, all of which retain the C-terminal lectin domain common to i-CD23, occur sequentially via initial formation of the 37 kDa fragment (Letellier et al, J Exp Med, 172 [1990] 693-700). An alternative intracellular cleavage pathway leads to a stable 16 kDa fragment differing in the C-terminal domain from i-CD23 (Grenier-Brosette et al, Eur J Immunol, 22 [1992] 1573-1577).

Several activities have been ascribed to membrane bound i-CD23 in humans, all of which have been shown to play a role in IgE regulation. Particular activities include: a) antigen presentation, b) IgE mediated eosinophil cytotoxicity, c) B cell homing to germinal centres of lymph nodes and spleen, and d) downregulation of IgF, synthesis (Delespesse et al, Adv Immunol, 49 [1991] 149-191). The three higher molecular weight soluble CD23 fragments (Mr 37, 33 and 29 kDa) have multifunctional cytokine properties which appear to play a major pole in IgE production. Thus, the excessive formation of s-CD23 has been implicated in the overproduction of IgE, the hallmark of allergic diseases such as extrinsic asthma, rhinitis, allergic conjunctivitis, eczema, atopic dermatitis and anaphylaxis (Sutton and Gould, Nature, 366, [1993] 421-428).

Other biological activities attributed to s-CD23 include the stimulation of B cell growth and the induction of the release of mediators from monocytes. Thus, elevated levels of s-CD23 have been observed in the serum of patients having B-chronic lymphocytic leukaemia (Sarfati et al, Blood, 71 [1988] 94-98) and in the synovial fluids of patients with rheumatoid arthritis (Chomarat et al, Arthitis and Rheumatism, 36 [1993] 234-242). That there is a role for CD23 in inflammation is suggested by a number of sources. First, sCD23 has been reported to bind to extracellular receptors which when activated are involved in cell-mediated events of inflammation. Thus, sCD23 is reported to directly activate monocyte TNF, IL-1, and IL-6 release (Armant et al, vol 180, J. Exp. Med., 1005-1011 (1994)). CD23 has been reported to interact with the B2-integrin adhesion molecules, CD11b and CD11c on monocyte/macrophage (S. Lecoanet-Henchoz et al, Immunity, vol 3; 119-125 (1995)) which trigger NO2⁻, hydrogen peroxide and cytokine (IL-1, IL-6, and TNF) release. Finally, IL-4 or IFN induce the expression of CD23 and its release as sCD23 by human monocytes. Ligation of the membrane bound CD23 receptor with IgE/anti-IgE immune complexes or anti CD23 mAb activates cAMP and IL5 production and thromboxane B2 formation, demonstrating a receptor-mediated role of CD23 in inflammation.

Because of these various properties of CD23, compounds which inhibit the formation of s-CD23 should have twofold actions of a) enhancing negative feedback inhibition of IgE synthesis by maintaining levels of i-CD23 on the surface of B cells, and b) inhibiting the immunostimulatory cytokine activities of higher molecular weight soluble fragments (Mr 37, 33 and 29 kDa) of s-CD23. In addition, inhibition of CD23 cleavage should mitigate sCD23-induced monocyte activation and mediator formation, thereby reducing the inflammatory response.

TNFα is a pro-inflammatory cytokine which is released from stimulated cells by specific cleavage of a 76-amino acid signal sequence in the inactive precursor to generate the mature form. The cleavage of TNFα has been reported to be carried out by a metalloprotease (Gearing, A. J. H. et al, (1994) Nature 370, 555-557; McGeehan, G. M. et al, (1994) Nature 370, 558-561; Mohler, K. M. et al, (1994) Nature 370, 218-220). Compounds reported to inhibit the cleavage of TNFα by the TNF processing enzyme can be broadly described as matrix metalloprotease inhibitors, particularly of the hydroxamic acid class.

TNFα is induced in a variety of cell types in response to bacteria, endotoxin, various viruses and parasites, so that one physiological function ascribed to TNFα is a contribution to the inflammatory response to acute infection by bacteria, parasites, etc (Dinarello, C. A. (1992) Immunol. 4, 133-145). Overproduction of TNFα has been implicated in disease states such as rheumatoid arthritis, septic shock, Crohn's disease and cachexia (Dinarello, 1992). Inhibition of processing of TNFα to the mature, active form would therefore be beneficial in the treatment of these inflammatory disorders. TNFα may also contribute to the destruction of tissue in autoimmune disease although it is not an initiating factor in these diseases. Confirming the importance of TNFα in rheumatoid arthritis, TNFα antibodies have been shown to reduce the severity of disease in short term studies in rheumatoid arthritis models (Elliott, M. J., et al (1993) Arthrit. Rheun 12, 1681-1690; Elliott et al (1994) Lancet 344, 1125-1127).

International Patent Application No. WO 97/27174 (Shionogi & Co., Ltd) and International Patent Application number WO 95/35275 (British Biotech Ltd) disclose that certain compounds of formula (A):

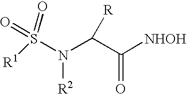

wherein R¹ is arylalkyl or heteroarylalkyl are effective inhibitors of metalloproteinases.

International Patent Application No. WO 98/46563 (British Biotech Ltd) discloses that certain compounds of formula (A) above in which R¹ is phenylalkyl or heteroarylalkyl are effective inhibitors of matrix metalloproteases.

According to the present invention, there is provided a compound of formula (I):

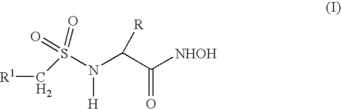

wherein

R is hydrogen, alkyl, alkenyl, alkynyl, aryl, heteroaryl or heterocyclyl; and

R¹ is bicyclyl or heterobicyclyl.

Alkyl, alkenyl and alkynyl groups referred to herein in the definition of the R group include straight, branched and cyclic groups containing up to eight carbon atoms, and are optionally substituted by one or more groups selected from the group consisting of aryl, heterocyclyl, (C1-6)alkylthio, (C2-6)alkenylthio, (C2-6)alkynylthio, aryloxy, arylthio, heterocyclyloxy, heterocyclylthio, (C1-6)alkoxy, (C1-6)alkenyloxy, (C1-6)alkynyloxy, aryl(C1-6)alkoxy, aryl(C1-6)alkylthio, amino, mono- or di-(C1-6)alkylamino, acylamino, sulfonylamino including (C1-6)alkylsulfonylamino, aryl(C1-6)alkyl sulfonylamino, aryl(C1-6)alkenylsulfonylamino and arylsulfonylamino, cycloalkyl, cycloalkenyl, carboxylic acid (C1-6) esters, hydroxy, halogen and carboxamide: CONR²R³ where R² and R³ are independently selected from the group consisting of hydrogen, alkyl, aryl, arylalkyl and heterocyclyl, and includes R² and R³ as part of a heterocyclyl group.

Cycloalkyl and cycloalkenyl groups referred to herein in the definition of the R group include groups having between three and eight ring carbon atoms and are optionally substituted as described hereinabove for alkyl, alkenyl and alkynyl groups.

When used herein in the definition of the R group, the term “aryl” includes phenyl. Suitably any aryl group, including phenyl, may be optionally substituted by up to five, preferably up to three substituents. Suitable substituents include halogen, CF₃, OCF₃, CN, (C₁₋₆)alkyl, (C₁₋₆)alkoxy, hydroxy, amino, heterocyclyl, heterocyclyl(C1-6)alkyl, mono- and di-N-(C1-6)alkylamino, acylamino, acyloxy, carboxy, (C1-6)alkoxycarbonyl, aminocarbonyl, mono- and di-N-(C1-6)alkylaminocarbonyl, mono- and di-N-(C1-6)alkylaminoalkyl, aminosulfonyl, (C1-6)alkylsulfonyl, mono- and di-N-(C1-6)alkylaminosulfonyl, (C1-6)mono- and dialkylaminosulfonyloxy, (C1-6)alkylsulfonyloxy, haloalkylsulfonyloxy including trifluoromethylsulfonyloxy, (C1-6)alkylthio and (C1-6)alkylsulfonylamino optionally substituted by alkyl. The term “aryl” includes single and fused rings, of which at least one is aromatic, which rings may be unsubstituted or substituted by, for example, up to three substituents as set out above. Each ring suitably has from 4 to 7, preferably 5 or 6, ring atoms.

When used herein in the definition of the R group the term “heteroaryl” suitably includes any heterocyclyl group which incorporates at least one aromatic ring (heterocyclic or carbocyclic). Suitable heteroaryl groups include thiophene, such as thiophen-2-yl, thiophen-3-yl and benzothiophen-3-yl.

When used herein in the definition of the R group the terms “heterocyclyl” and “heterocyclic” suitably include, unless otherwise defined, aromatic and non-aromatic, single and fused, rings suitably containing up to four heteroatoms in each ring, each of which is selected from oxygen, nitrogen and sulphur, which rings, may be unsubstituted or substituted by, for example, up to three substituents. Each ring suitably has from 4 to 7, preferably 5 or 6, ring atoms. A fused heterocyclic ring system may include carbocyclic rings and need include only one heterocyclic ring. Preferably a substituent for a heterocyclyl group is selected from halogen, (C1-6)alkyl, (C1-6)alkoxy, hydroxy, CF₃, OCF₃, CN, amino, mono-and di-N-(C1-6)alkylamino, acylamino, acyloxy, carboxy, (C1-6)alkoxycarbonyl, aminocarbonyl, mono- and di-N-(C1-6)alkylaminocarbonyl, (C1-6)alkylsulfonylamino, aminosulfonyl, (C1-6)alkylthio and (C1-6)alkylsulfonyl.

When used herein in the definition of the R¹ group “bicyclyl” means fused bicyclic rings suitably containing 4 to 7, preferably 5 or 6 ring atoms in each ring. One ring of the bicyclyl may be saturated or partially saturated. Suitable bicyclyl groups include naphthyl such as 2-naphthyl, tetrahydronaphthyl such as 2-tetrahydronaphthyl, and indanyl such as 2-indanyl.

When used herein in the definition of the R¹ group, heterobicyclyl means fused bicyclic aromatic and non-aromatic rings containing up to 4 heteroatoms in each ring, each of which is selected from oxygen, nitrogen and sulphur. Each ring suitably has from 4 to 7, preferably 5 or 6, ring atoms. The fused bicyclic ring system may include one carbocyclic ring and one of the rings may be saturated or partially saturated. Suitable heterobicyclyl groups include benzothiophene such as benzothiophen-5-yl and benzothiophen-6-yl.

Aromatic rings in bicyclyl and heterobicyclyl ring systems may be optionally substituted with up to three substituents. Suitable substituents include fluorine.

In a particular aspect of the invention, R is isobutyl, phenyl, 4-hydroxyphenyl, 4-fluorophenyl, 4-isopropylphenyl, 3-fluorophenyl, indol-3-ylmethyl, benzothiophen-3-yl, benzothiophen-3-ylmethyl, 4-trifluoromethoxyphenyl, 4-trifluoromethanesulfonyloxyphenyl, phenylmethanesulfonylaminomethyl, phenethyl or pthalimidomethyl and/or R¹ is 2-naphthyl, (R)-1,2,3,4-tetrahydronaphthalen-2-yl, benzothiophen-5-yl optionally substituted by fluorine, benzothiophen-6-yl or indan-2-yl. In a further aspect of the invention, R and R¹ are selected from the group consisting of the values ascribed to it in the Examples hereinbelow. Preferably, the compound of formula (I) of the invention is selected from the group consisting of the compounds described in the Examples hereinbelow.

According to a further aspect, the present invention provides the use of a compound of formula (I) for the production of a medicament for the treatment or prophylaxis of disorders such as allergy, inflammatory disorders, and autoimmune disease, in which the overproduction of s-CD23 is implicated.

In a further aspect the invention provides a method for the treatment or prophylaxis of disorders such as allergy, inflammatory disorders, and autoimmune disease, in which the overproduction of s-CD23 is implicated, which method comprises the administration of a compound of formula (1), to a human or non-human mammal in need thereof.

The invention also provides a pharmaceutical composition for the treatment or prophylaxis of disorders such as allergy, inflammatory disorders, and autoimmune disease, in which the overproduction of s-CD23 is implicated which comprises a compound of formula (I) and optionally a pharmaceutically acceptable carrier therefor.

Particular inflammatory disorders include CNS disorders such as Alzheimer's disease, multiple sclerosis, and multi-infarct dementia, as well as the inflammation mediated sequel of stroke and head trauma.

According to a further aspect, the present invention provides the use of a compound of formula (I) for the production of a medicament for the treatment or prophylaxis of conditions mediated by TNF, including, but not limited to, inflammation, fever, cardiovascular effects, haemorrhage, coagulation and acute phase response, cachexia and anorexia, acute infections, shock states, graft versus host reactions and autoimmune disease.

In a further aspect the invention provides a method for the treatment or prophylaxis of conditions mediated by TNF, which method comprises the administration of a compound of formula (I), to a human or non-human mammal in need thereof.

The invention also provides a pharmaceutical composition for the treatment or prophylaxis of conditions mediated by TNF, which comprises a compound of formula (I) and optionally a pharmaceutically acceptable carrier therefor.

The present inventors have surprisingly found that the compounds of the invention are potent and selective inhibitors of both CD23 processing and TNF processing, whilst having little or no activity as inhibitors of matrix metalloproteases.

It is to be understood that the pharmaceutically acceptable salts, solvates and other pharmaceutically acceptable derivatives of the compound of formula (I) are also included in the present invention.

Salts of compounds of formula (I) include for example acid addition salts derived from inorganic or organic acids, such as hydrochlorides, hydrobromides, hydroiodides, p-toluenesulphonates, phosphates, sulphates, acetates, trifluoroacetates, propionates, citrates, maleates, fumarates, malonates, succinates, lactates, oxalates, tartrates and benzoates.

Salts may also be formed with bases. Such salts include salts derived from inorganic or organic bases, for example alkali metal salts such as sodium or potassium salts, and organic amine salts such as morpholine, piperidine, dimethylamine or diethylamine salts.

The compounds of the invention may be prepared by use of any appropriate conventional method, for example by analogy with the methods disclosed in patent publication EP-A-0 606 046.

Accordingly, a further aspect of the invention provides a process for preparing a compound of formula (I) as defined hereinabove, which process comprises:

(a) deprotecting a compound of formula (II):

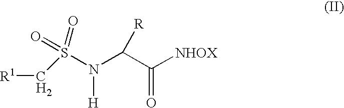

wherein R and R¹ are as defined hereinabove, and X is a protecting group such as benzyl, t-butyldimethylsilyl or trimethylsilyl, or

(b) reacting a compound of formula (m):

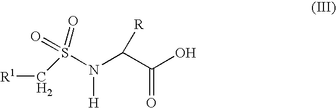

wherein R and R¹ are as defined hereinabove, with hydroxylamine or a salt thereof, or

(c) converting a compound of formula (I) to a different compound of formula (I) as defined hereinabove.

Compounds of formula (II) and (E) are novel and form a further aspect of the invention.

Compounds of formula (II) can be prepared from compounds of formula (I) by reaction with a protected hydroxylamine. Compounds of formula (III) can be prepared by deprotection of a compound of formula (I):

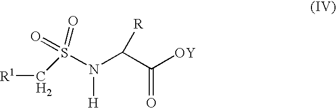

wherein R and R¹ are as defined hereinabove, and Y is a protecting group such as t-butyl or trimethylsilyl.

Suitable protecting groups for a hydroxamic acid are well known in the art and include benzyl, trimethylsilyl, t-butyl and t-butyldimethylsilyl.

Suitable protecting groups for a carboxylic acid are well known in the art and include t-butyl, benzyl, methyl and trimethylsilyl.

Compounds of formula (IV) can be prepared by reacting a compound of formula (V):

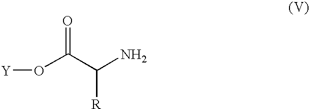

wherein R and Y are as defined hereinabove, with a compound of formula (VI):

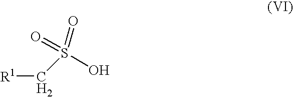

wherein R and R¹ are as defined hereinabove, or an activated derivative thereof, such as a sulfonyl chloride compound of formula (VIa):

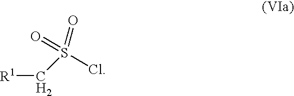

If in the compound of formula (V) Y is trimethylsilyl the protecting group may be introduced by treatment of a carboxylic acid of formula (VII):

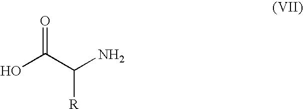

with a suitable silylating agent, e.g. BSTFA, and reacted in situ with an activated derivative of a compound of formula (VI) such as the sulfonyl chloride.

Sulfonylchlorides of formula (VIa) can be prepared by first reacting a compound of formula (VIII):

R¹—CH₂-Z (VIII)

wherein R¹ is as described hereinabove and Z is halogen or an alkyl or aryl sulfonate with sodium sulfite to give the corresponding sodium sulfonate, which can optionally be converted by tetra-n-butyl ammonium hydrogen sulfate into the corresponding tetra-n-butylammonium sulfonate salt. The tetra-n-butylammonium sulfonate salt may be formed by direct conversion of a compound of formula (VIII) where Z is preferably bromide, chloride or iodide under phase transfer conditions. However, phase transfer catalysis under normal conditions (i.e. the ratio of phase transfer cations to compound of formula (VIII) is less than 1:1) in this system results in a poor yield of product since as soon as sulfonate and bromide ions begin to be formed they sequester the catalytic cation into the organic phase and the reaction ceases. It has been found that improved yields are achieved when more than one equivalent of the phase transfer cation is used, i.e. the ratio of phase transfer cation to compound of formula (VIII) is >1:1. Accordingly, a further aspect of the invention is a process for preparing a compound of formula (VIa) comprising converting a compound of formula (VIII) into a compound of formula (IX):

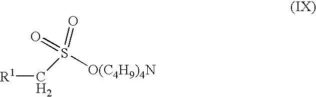

in which R¹ is as hereinbefore defined under phase transfer conditions in which the ratio of tetra-n-butylammonium cations to compound of formula (VI) is greater than 1:1. Preferably, about two equivalents of a phase transfer cation are used to allow complete reaction to take place, i.e. the ratio of phase transfer cation to compound of formula (VIII) is about 2:1.

Conversion of the sulfonate salt into the sulfonyl chloride may be achieved using phosphorus oxychloride in acetonitrile and tetrahydrothiophene-1,1-dioxide at elevated temperature (Abdellaoui et al, Synth. Commun. 1995, 25(9) 1303). In the case of the tetra-n-butylammonium sulfonate the sulfonyl chloride is prepared using a chlorinating agent such as phosphorus pentachloride or triphosgene, preferably under low temperature conditions such as −20° C. or below, and preferably by addition of the sulfonate salt to the chlorinating agent.

The starting materials and other reagents are available commercially or can be synthesised by well-known and conventional methods. Non natural amino acid precursors can be prepared using methods described in the literature. For example, the preparation of (R)-2-tert-butoxycarbonylamino-3-aminopropionic acid is described in: Zhang, Kauffman, Pesti, Yin, J. Org. Chem. 1997, 62, 6918.

Alpha aryl glycines can be prepared using the methodology described in Petasis, Goodman, Zavialov, Tetrahedron, 1997, 53, 16463. Alternative methodology for preparing aryl glycines is reviewed in Zaugg, Synthesis, 1984, 85. Substituted aryl glycines may be prepared from the corresponding triflates or halides using the methodogy described by Buchwald (eg Wolfe, Tomori, Sadighi, Yin, Buchwald, J. Org. Chem., 2000, 65, 1158).

β-lactones derived from serine, eg N-(tert-butoxycarbonyl)-serine β-lactone, can be ring opened with a range of heterocycles to give non natural amino acid derivatives (Pansare, Arnold, Vederas, Org. Syn. 1992, 70, 10). For example, reaction with pyrazoles affords substituted β-(pyrazol-1-yl)-alanine derivatives.

Functional group modifications required for the preparation of amino acid precursors may require appropriate protection. For example the introduction of N-trityl protection with in situ silylation of hydroxyl groups is described in Dolence, Lin, Miller, J. Med. Chem. 1991, 34, 956. Selective protection of carboxyl groups as tert-butyl esters in the presence of hydroxyl group's may be achieved using O-tert-butyl N,N′-diisopropylisourea (Maguire et al, J. Org. Chem., 1990, 55, 948).

The isomers, including stereoisomers, of the compounds of the present invention may be prepared as mixtures of such isomers or as individual isomers. The individual isomers may be prepared by any appropriate method, for example individual stereoisomers may be prepared by stereospecific chemical synthesis starting from chiral substrates or by separating mixtures of diastereoisomers using known methods. Racemates may be separated at any suitable stage of the synthesis. Chiral preparative HPLC methods may be used to separate final products. Enzymic resolution may be carried out on appropriate substrates. For example, arylglycines may be separated into single enantiomers by selective hydrolysis of the corresponding N-formyl derivatives with acylase I.

In a preferred aspect, the invention provides compounds of formula (IA):

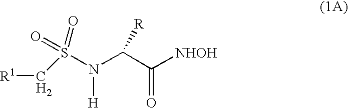

It is preferred that the compounds are isolated in substantially pure form.

As stated herein an inhibitor of the formation of soluble human CD23 has useful medical properties. Preferably the active compounds are administered as pharmaceutically acceptable compositions.

The compositions are preferably adapted for oral administration. However, they may be adapted for other modes of administration, for example in the form of a spray, aerosol or other conventional method for inhalation, for treating respiratory tract disorders; or parenteral administration for patients suffering from heart failure. Other alternative modes of administration include sublingual or transdermal administration.

The compositions may be in the form of tablets, capsules, powders, granules, lozenges, suppositories, reconstitutable powders, or liquid preparations, such as oral or sterile parenteral solutions or suspensions.

In order to obtain consistency of administration it is preferred that a composition of the invention is in the form of a unit dose.

Unit dose presentation forms for oral administration may be tablets and capsules and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulphate.

The solid oral compositions may be prepared by conventional methods of blending, filling or tabletting. Repeated blending operations may be used to distribute the active agent throughout those compositions employing large quantities of fillers. Such operations are of course conventional in the art. The tablets may be coated according to methods well known in normal pharmaceutical practice, in particular with an enteric coating.

Oral liquid preparations may be in the form of, for example, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring-agents.

For parenteral administration, fluid unit dosage forms are prepared utilising the compound and a sterile vehicle, and, depending on the concentration used, can be either suspended or dissolved in the vehicle. In preparing solutions the compound can be dissolved in water for injection and filter sterilised before filling into a suitable vial or ampoule and sealing. Advantageously, adjuvants such as a local anaesthetic, a preservative and buffering agents can be dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspending in the sterile vehicle. Advantageously, a surfactant or wetting agent is included in the composition to facilitate uniform distribution of the compound.

Compositions of this invention may also suitably be presented for administration to the respiratory tract as a snuff or an aerosol or solution for a nebulizer, or as a microfine powder for insufflation, alone or in combination with an inert carrier such as lactose. In such a case the particles of active compound suitably have diameters of less than 50 microns, preferably less than 10 microns for example diameters in the range of 1-50 microns, 1-10 microns or 1-5 microns. Where appropriate, small amounts of other anti-asthmatics and bronchodilators, for example sympathomimetic amines such as isoprenaline, isoetharine, salbutamol, phenylephrine and ephedrine; xanthine derivatives such as theophylline and aminophylline and corticosteroids such as prednisolone and adrenal stimulants such as ACTH may be included.

The compositions may contain from 0.1% to 99% by weight, preferably from 10-60% by weight, of the active material, depending upon the method of administration. A preferred range for inhaled administration is 10-99%, especially 60-99%, for example 90, 95 or 99%.

Microfine powder formulations may suitably be administered in an aerosol as a metered dose or by means of a suitable breath-activated device.

Suitable metered dose aerosol formulations comprise conventional propellants, cosolvents, such as ethanol, surfactants such as oleyl alcohol, lubricants such as oleyl alcohol, desiccants such as calcium sulphate and density modifiers such as sodium chloride.

Suitable solutions for a nebulizer are isotonic sterilised solutions, optionally buffered, at for example between pH 4-7, containing up to 20 mg/ml of compound but more generally 0.1 to 10 mg/ml, for use with standard nebulisation equipment.

An effective amount will depend on the relative efficacy of the compounds of the present invention, the severity of the disorder being treated and the weight of the sufferer. Suitably, a unit dose form of a composition of the invention may contain from 0.1 to 1000 mg of a compound of the invention (0.001 to 10 mg via inhalation) and more usually from 1 to 500 mg, for example 1 to 25 or 5 to 500 mg. Such compositions may be administered from 1 to 6 times a day, more usually from 2 to 4 times a day, in a manner such that the daily dose is from 1 mg to 1 g for a 70 kg human adult and more particularly from 5 to 500 mg. That is in the range of about 1.4×10 m2 mg/kg/day to 14 mg/kg/day and more particularly in the range of about 7×10-2 mg/kg/day to 7 mg/kg/day.

The following examples illustrate the invention but do not limit it in any way.

Biological Test Methods

Procedure 1: The ability of test compounds to inhibit the release of soluble CD23 was investigated by use of the following procedure.

RPMI 8866 Cell Membrane CD23 Cleavage Activity Assay:

Plasma membranes from RPMI 8866 cells, a human Epstein-Barr virus transformed B-cell line (Sarfati et al., Immunology 60 [1987] 539-547) expressing high levels of CD23 are purified using an aqueous extraction method. Cells resuspended in homogenization buffer (20 mM HEPES pH 7.4, 150 mM NaCl, 1.5 mM MgCl2, 1 mM DTT) are broken by N₂ cavitation in a Parr bomb and the plasma membrane fraction mixed with other membranes is recovered by centrifugation at 10,000×g. The light pellet is resuspended in 0.2 M potassium phosphate, pH 7.2 using 2 ml per 1-3 g wet cells and the nuclear pellet is discarded. The membranes are further fractionated by partitioning between Dextran 500 (6.4% w/w) and polyethylene glycol (PEG) 5000 (6.4% w/w) (ref), at 0.25 M sucrose in a total of 16 g per 10-15 mg membrane proteins [Morre and Morre, BioTechniques 7, 946-957 (1989)]. The phases are separated by brief centrifugation at 1000×g and the PEG (upper) phase is collected, diluted 3-5 fold with 20 mM potassium phosphate buffer pH 7.4, and centrifuged at 100,000×g to recover membranes in that phase. The pellet is resuspended in phosphate-buffered saline and consists of 34 fold enriched plasma membranes as well as some other cell membranes (e.g. lysosomes, Golgi). The membranes are aliquoted and stored at −80° C. Fractionation at 6.6% Dextran/EG yields plasma membranes enriched 10-fold.

The fractionated membranes are incubated at 37° C. for times up to 4 hrs to produce fragments of CD23 which are separated from the membrane by filtration in 0.2 micron Durapore filter plates (Millipore) after quenching the assay with 5 uM Preparation 1 from P 30994. sCD23 released from the membrane is determined using the EIA kit from The Binding Site (Birmingham, UK) or a similar one utilising MHM6 anti-CD23 mAb [Rowe et al., Int. J. Cancer, 29, 373-382 (1982)] or another anti-CD23 mAb as the capture antibody in a sandwich EIA. The amount of soluble CD23 made by 0.5 ug membrane protein in a total volume of 50 ul phosphate-buffered saline is measured by EIA and compared to the amount made in the presence of various concentrations of inhibitors. Inhibitors are prepared in solutions of water or dimethylsulfoxide (DMSO) and the final DMSO concentration is not more than 2%. IC50's are determined by curve fitting as the concentration where 50% inhibition of production of sCD23 is observed relative to the difference in sCD23 between controls incubated without inhibitor.

Results

The compounds of the Examples all showed IC₅₀ values of ≦1 uM.

Procedure 2: The ability of test compounds to inhibit collagenase was investigated using the following procedure.

Collagenase Inhibition Assay:

The potency of compounds to act as inhibitors of collagenase was determined by the method of Cawston and Barrett (Anal. Biochem. 99, 340-345, 1979), hereby incorporated by reference, whereby a 1 mM solution of the inhibitor being tested or dilutions thereof, was incubated at 37° C. for 18 h with collagen and human recombinant collagenase, from synovial fibroblasts cloned, expressed and purified from E. Coli, (buffered with 150 mM Tris, pH 7.6, containing 15 mM calcium chloride, 0.05% Brij 35; 200 mM sodium chloride and 0.02% sodium azide). The collagen was acetylated ³H type 1 bovine collagen prepared by the method of Cawston and Murphy (methods in Enzymology 80, 711, 1981) The samples were centrifuged to sediment undigested collagen and an aliquot of the radioactive supernatant removed for assay on a scintillation counter as a measure of hydrolysis. The collagenase activity in the presence of 1 mM inhibitor, or dilution thereof, was compared to activity in a control devoid of inhibitor and the results reported as that concentration effecting 50% of the collagenase (IC₅₀).

Results

The compounds of Examples 5, 6, 9, 12, 14, 27, 28, 29, 31, 33, 38, 42, 43, 56 and 69 showed IC₅₀ values of ≧10 uM

Preparation of Intermediates

Preparation 1: Naphthalen-2-ylmethanesulfonyl chloride

Step 1: Sodium naphthalen-2-ylmethanesulfonate—2-Bromomethyl-naphthalene (70 g), was dissolved in dioxan(350 ml) and treated with sodium sulfite (240 g) in water (500 ml). The mixture was heated under reflux for 30 min. On cooling a white solid was obtained which was filtered off, washed with ether and dried to give the subtitle methanesulfonate salt (69 g).

Step 2: Naphthalen-2-ylmethanesulfonyl chloride—To sodium naphthalen-2-ylmethanesulfonate (12 g) in tetrahydrothiophene-1,1-dioxide (96 ml) were added acetonitrile (48 ml) and phosphorus oxychloride (24 ml) and the mixture was heated. When the internal temperature reached 100° C. unreacted starting material was filtered off and the hot filtrate was poured onto ice. A brown solid was filtered off and washed with hexane to give title compound (5.5 g).

Preparation 2: Benzo[b]thiophene-5-methanesulfonyl chloride

Step 1: 5-Bromomethylbenzo[b]thiophene—A solution containing 5-methylbenzo[b]thiophene (37 g), N-bromosuccinimide (46 g), and tetrachloromethane (40 ml) was refluxed for 4 h, cooled, and filtered. The filtrate was evaporated and the resultant residue crystallised from hexane to give the subtitle compound (40 g).

Step 2: Tetra-n-butylammonium benzo[b]thiophene-5-methanesulfonate—A mixture containing 5-bromomethylbenzo[b]thiophene (40 g), tetra-n-butylammonium hydrogen sulfate (135 g), sodium hydroxide (14 g), sodium sulfite (45 g), dichloromethane (300 ml), and water (300 ml) was stirred vigorously overnight. The organic layer was dried MgSO₄), evaporated, dissolved in THF (130 ml), re-evaporated, and dissolved again in THF (130 ml). Addition of ether (200 ml) gave the crystalline subtitle compound containing an equimolar amount of tetra-n-butylammonium bromide (132 g).

Step 3: Benzo[b]thiophene-5-methanesulfonyl cloride—A solution of the tetra-n-butylammonium benzo[b]thiophene-5-methanesulfonate from step 2 (30 g) in dichloromethane (150 ml) was added to a cooled suspension of phosphorus pentachloride (83 g) in dichloromethane (150 ml) at an internal temperature of −20° C. The solution was warmed to room temperature and maintained at room temperature for 15 min, then filtered through a pad of silicagel washing with ethyl acetate:hexane (1:1). The combined eluates were dissolved in toluene, and the resulting solution again filtered through silica gel, eluting with more toluene. Evaporation of the eluate and crystallisation from hexane gave the title compound (7.5 g). ¹H NMR 8(CDCl₃) 7.95(1H,d,J 8 Hz), 7.94(1H,s), 754(1H,d,J 6 Hz), 7.43(1H,d,J 8 Hz), 7.38(1H,d,J 6 Hz), 4.99(2H,s). In like manner was prepared naphthalene-2-methanesulfonyl chloride from 2-bromomethylnaphthalene.

Preparation 3: Indane-2-methanesulfonyl chloride

Step 1: 2-Bromomethylindane—A solution of 2-hydroxymethylindane (1.0 g) in dichloromethane (25 ml) at 0° C. was treated with triethylamine (11.0 ml) and methanesulfonyl chloride (0.6 ml). After 30 min the solution was washed with hydrochloric acid, aqueous sodium hydrogen carbonate, and brine, dried (MgSO₄), and evaporated. The resulting crude mesylate was dissolved in acetone (25 ml) and lithium bromide (1.8 g) added. After refluxing overnight the mixture was cooled and filtered, and the filtrate evaporated then partitioned between water and hexane. The hexane layer was filtered through silicagel and evaporated to provide the subtitle compound (0.49 g).

Step 2: Tetra-n-butylammonium indane-2-methanesulfonate—A mixture of 2-bromomethylindane (0.49 g), sodium sulfite (0.32 g), ethanol (5 ml), and water (10 ml) was refluxed overnight then cooled, and tetra-n-butylammonium hydrogen sulfate (0.80 g), sodium hydroxide (0.09 g), dichloromethane (20 ml), and water (20 ml) were added. After stirring the mixture for 10 min the organic layer was separated, dried (MgSO₄) and evaporated to give the subtitle compound (0.95 g).

Step 3: Indane-2-methanesulfonyl chloride—A solution of tetra-n-butylammonium indane-2-methanesulfonate (0.90 g) in dichloromethane (20 ml) was treated with DMF (1 drop) and triphosgene (0.30 g). After 2 h, the solution was evaporated and the residue filtered through silicagel eluting with ethyl acetate:hexane (1:1) to give the title compound (0.32 g). In like manner were prepared R-(1,2,3,4-tetrahydronaphthalen-2-yl)methanesulfonyl chloride from R-2-hydroxymethyl-1,2,3,4-tetrhydronaphthalene; benzo[b]thiophene-5-methanesulfonyl chloride from 5-bromomethylbenzo[b]thiophene, and naphthalene-2-methanesulfonyl chloride from. tetra-n-butylammonium naphthalene-2-methanesulfonate.

Preparation 4: Benzo[b]thiophene-6-methanesulfonyl chloride

Step 1: 6-Hydroxymethylbenzo[b]thiophene—A solution of benzo[b]thiophene-carboxylic acid (1.3 g) in THF (10 ml) at 0° C. was treated with lithium aluminium hydride (30 ml, 1M in THE). After 1 h at 0° C. and 1 h at room temperature, excess ethyl acetate was added and the reaction mixture then partitioned between hydrochloric acid and ether. The organic layer was dried (MgSO₄) and evaporated and the residue crystallised from hexane:ethyl acetate to give the subtitle compound (0.7 g).

Step 2: 6-Bromomethylbenzo[b]thiophene—Phosphorus oxybromide (2.7 g) was added to a solution of 6-hydroxymethylbenzo[b]thiophene (0.7 g) in ether (50 ml) at reflux. After 3 h at reflux the solution was washed with water, aqueous sodium hydrogen carbonate, and brine, dried (MgSO₄) and evaporated to give the title compound (0.7 g).

Step 3: Tetra-n-butylammonium benzo[b]thiophene-6-methanesulfonate—was prepared according to the method of Preparation 2, step 2.

Step 4: Benzo[b]thiophene-6-methanesulfonyl chloride—was prepared according to the method of Preparation 2, step 3.

Preparation 5: 2-Fluorobenzo[b]thiophene-5-methanesulfonyl chloride

Step 1: 2-Fluoro-5-methylbenzo[b]thiopbene—A solution of 5-methylbenzo[b]thiophene (4.0 g) in diethyl ether (50 ml) at −10° C. was treated with n-butyllithium (19 ml, 1.6M in hexane). After 1 h at −10° C. N-fluorobenzenesulfonimide (10.4 g) in THF (20 ml) was added. After 1 h at rt the mixture was partitioned between aqueous ammonium chloride and hexane, and the organic layer was dried (MgSO₄), evaporated and chromatographed (silica gel, hexane) to give the subtitle compound (1.9 g).

Step 2: 2-Fluorobenzo[b]thiophene-5-methanesulfonyl chloride—Prepared from 2-fluoro-5-methylbenzo[b]thiophene using the methods described in Preparation 2.

Preparation 6: 3-Fluorobenzo[b]thiophene-5-methanesulfonyl chloride

Step 1: 5-Methylbenzo[b]thiophene-2-carboxylic acid—A solution of 5-methylbenzo[b]thiophene (6.1 g) in diethyl ether (50 ml) at −10° C. was treated with n-butyllithium (25 ml, 1.6M in hexane). After 1 h at −10° C. the solution was poured onto solid carbon dioxide then left to evaporate. Water and diethyl ether were then added. The aqueous layer was acidified with dilute hydrochloric acid and extracted with more diethyl ether to give the subtitle compound (5.4 g).

Step 2: 5-Methyl-3-fluorobenzo[b]thiophene-2-carboxylic acid—A solution of 5-methylbenzo[b]thiophene-2-carboxylic acid (1.0 g) in THE (15 ml) at −70° C. was treated with n-butyllithium (7.0 ml, 1.6M in hexane). After 1 h at −70° C. N-fluorobenzenesulfonimide (2.4 g) in THF (5 ml) was added. After 1 h at rt the mixture was partitioned between dilute hydrochloric acid and diethyl ether. The organic layer was dried (MgSO₄) and evaporated and the residue crystallised from dichloromethane to give the subtitle compound (0.750).

Step 3: 3-Fluoro-5-methylbenzo[b]thiophene—A mixture of 5-methyl-3-fluorobenzo[b]thiophene-2-carboxylic acid (0.75 g), copper powder (0.50 g), and quinoline (5 ml) was heated at 180° C. for 30 min then cooled and partitioned between dilute hydrochloric acid and hexane. The organic layer was dried (MgSO₄) and evaporated and chromatographed (silica gel, hexane) to give the subtitle compound (1.9 g).

Step 4: 3-Fluorobenzo[b]thiophene-5-methanesulfonyl chloride—Prepared from 3-fluoro-5-methylbenzo[b]thiophene using the methods described in Preparation 2.

Preparation 7: 6-Fluorobenzo[b]thiophene-5-methanesulfonyl chloride

Step 1: 2,4-Difluoro-3-trimethylsilyltoluene—To a solution of diisopropylamine (7.5 ml) in THF (100 ml) at 0° C. was added n-butyllithium (31 ml, 1.6M in hexane). After 10 min at −10° C. the solution was cooled to −70° C. and 2,4-difluorotoluene (5.7 ml) added, and after a further 1 h at −70° C. chlorotrimethylsilane (6.9 ml) was added. After 1 h at rt the mixture was partitioned between aqueous ammonium chloride and hexane, and the organic layer was dried (MgSO₄) and evaporated to give the subtitle compound (8.8 g).

Step 2: 2,4-Difluoro-3-trimethylsilyl-5-methylbenzadehyde—A solution of 2,4-difluoro-3-trimethylsilyltoluene (11.0 g) in THF (10 ml) at −70° C. was treated with TMEDA (0.8 ml) and s-butyllithium (4.0 ml, 13M in cyclohexane). After 1 h at −70° C. DMF (0.4 ml) was added followed by acetic acid then water. The mixture was partitioned between aqueous citric acid and diethyl ether. The organic layer was dried (MgSO₄) and evaporated to give the subtitle compound (0.80 g).

Step 3: 2,4-Difluoro-3-trimethylsilyl-5-methylbenzylidenerhodanine—A mixture of 2,4-difluoro-3-trimethylsilyl-5-methylbenzaldehyde (0.9 g), rhodanine (0.5 g), sodium acetate (1.3 g), and acetic acid (5 ml) was refluxed for 1 h then cooled and poured into water. The subtitle compound separated as yellow rhombs (1.3 g).

Step 4: 2-Mercapto-3-(2,4-Difluoro-5-methylphenyl)-propenoic acid—A solution of 2,4-difluoro-3-trimethylsilyl-5-methylbenzylidenerhodanine (0.34 g) in aqueous sodium hydroxide (2.5M, 5 ml) was heated for 1 h at 70° C. then diluted with water and washed with dichloromethane, then acidified and extracted with diethyl ether. The organic layer was dried (MgSO₄) and evaporated to give the subtitle compound (0.17 g).

Step 5: 5-Methyl-6-fluorobenzo[b]thiophene-2-carboxylic add—To a solution of 2-mercapto-3-(2,4-difluoro-5-methylphenyl)propenoic acid (1.7 g) in DMSO (20 ml) was added potassium tert-butoxide (1.7 g). After 18 h at 60° C. the solution was partitioned between aqueous citric acid and diethyl ether. The organic layer was dried (MgSO₄) and evaporated to give the subtitle compound (1 g).

Step 6: 6-Fluoro-5-methylbenzo[b]thiophene—was prepared by the method of Preparation 6, step 3.

Step 7: 6-Fluorobenzo[b]thiophene-5-methanesulfonyl chloride—Prepared from 6-fluoro-5-methylbenzo[b]thiophene using the methods described in Preparation 2.

### EXAMPLES

### Example 1

### (R)-N-Hydroxy-4-Methyl-2-(naphthalen-2-ylmethanesufonylamino)-pentanoic acid amide

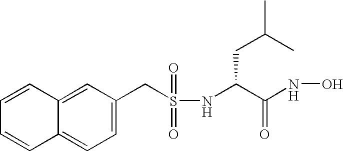

Step 1: (R)-4-Methyl-2-(naphthalen-2-ylmethanesulfonylamio)-pentanoic acid tert-butyl ester. To a mixture of (R)-2-amino-4-methyl-pentanoic acid tert-butyl ester hydrochloride salt (0.81 g, 3.62 mmol) and DMAP (0.32 g, 2.62 mmol) in pyridine (16 ml), at rt, under argon, was added portionwise naphthalen-2-yl-methanesulfonyl chloride (1.47 g, 6.27 mmol) over 5 min. The reaction was stirred overnight, then taken up into ethyl acetate (50 ml) and washed twice with 2N HCl (150 ml). The organic layer was washed with saturated sodium bicarbonate, water, dried (Na₂SO₄) and concentrated to give a crude product that was purified by chromatography (silica gel, step gradient 0-40% ethyl acetate/hexane) to give the subtitle compound as a white solid (0.61 g). MS electrospray (−ve ion), =390 (M−H⁻). ¹H N δ (CDCl₃): 7.88 (1H, m). 7.83 (3H, m), 7.55 (1H, m), 7.50 (2H, m), 4.63 (1H, d, J=9.1 Hz), 4.40 (2H, s), 3.86 (1H, m), 1.70 (1H, m), 1.46 (9H, s), 1.45 (2H, m), 0.87 (3H, d, J=6.5 Hz), 0.83 (3H, d, J=6.6 Hz).

Step 2: (R)-4-Methyl-2-(naphthalen-2-ylmethanesulfonylamino)-pentanoic acid. A solution of (R)-4-methyl-2-(naphthalen-2-ylmethanesulfonylamino)pentanoic acid tert-butyl ester (0-59 g, 1.50 mmol) in DCM/TFA (3:2) (5 ml) was stirred at room temperature for 4 h. The solvent was evaporated. Azeotroping with chloroform then toluene gave the subtitle compound (0.51 g). MS electrospray (+ve ion)=358 (M+Na⁺); MS electrospray (−ve ion) 334 (M−H⁻). ¹H NMR δ (CDCl₃): 7.83 (4H, m), 5.35 (3H, m), 4.68 (1H, d, J=9.1 Hz), 4.45 (2,H, s), 3.94 (1H, m), 1.66 (1H, m), 1.47 (2H, m), 0.84 (3H, d, J=65 Hz), 0.80 (3H, d, J=65 Hz). Acid proton not observed.

Step 3: (R)-N-Hydroxy-4-Methyl-2-(naphthalen-2-ylmethanesulfonylamino)pentanoic acid amide. A mixture of (R)-4-methyl-2-naphthalen-2-ylmethanesulfonylaminopetanoic acid (0.30 g, 0.89 mmol), EDC (0.34 g, 1.78 mmol), and HOAT (0.24 g, 1.78 mmol) was stirred in dry DMF (5 ml) at room temperature under argon for 10 min. Hydroxylamine hydrochloride (0.19 g, 2.67 mmol) and N-methylmorpholine (0.29 ml, 2.67 mmol) were added and the reaction mixture was stirred for 4 h. The solvent was evaporated and the residue was partitioned between ethyl acetate and water. The organic layer was washed with 10% citric acid, saturated sodium bicarbonate solution, saturated brine, dried (Na₂SO₄) and concentrated to give an orange solid. The crude compound was purified by preparative HPLC to give the title compound as a white solid (0.078 g). MS electrospray (+ve ion) 351 (M+H⁺), 373 (M+Na⁺). MS electrospray (−ve ion) 349 (M−H⁻). ¹H NMR δ (DMSO-d₆): 10.85 (1H, s), 9.00 (1H, s), 7.91 (4H, m), 7.52 (4H, m), 4.42 (1H, d, J=13.6 Hz), 4.35 (1H, d, J=13.6 Hz), 1.70 (1H, m), 1-50 (1H, m), 1.40 (2H t, J=7.3 Hz), 0.8 (3H, s), 0.77 (3H, s).

### Example 2

### (R)-N-Hydroxy-2-(naphthalen-2-ylmethanesulfonylamino)-2-phenyl-acetamide

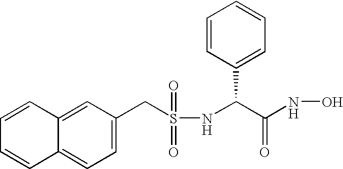

Step 1: (R)-(Naphthalen-2-ylmethanesulfonylamino)-2-phenyl-acetic acid—To (R) phenylglycine (0.245 g) was added DMF (1 ml), pyridine (1 ml) followed by BSTFA (0.86 ml). The mixture was warmed to 60° C. After 30 min the solution was cooled to 0° C. and a solution of naphthalene-2-yl-methanesulfonyl chloride (0.3 g) in DMF (1 ml) was added dropwise followed by the addition of Et₃N (0.175 ml). The reaction mixture was left at rt for 2 h. A polymer scavenger PS-Isocyanate (0.5 g) was added to the reaction mixture. After 10 min the scavenger was filtered off. Addition of aqueous potassium hydrogen sulfate to the filtrate produced a yellow solid that was filtered off and dried to give the subtitle compound (0.4 g).

Step 2: (R)-(Naphthalen-2-ylmethanesulfonylamino)-N-(t-butyldimethylsilyloxy)-2-phenyl-acetic acid—A solution of (R)-(naphthalen-2-ylmethanesulfonylamino)-2-phenyl-acetic acid (0.2 g) in dichloromethane (2 ml) was treated with O-t-butyldimethylsilylhydroxylamine (0.124 g) in dichloromethane (0.5 ml), and EDC-methiodide (0.249 g) at 0° C. After 2 h at rt the solution was treated with aqueous sodium bicarbonate. The organic layer was separated, dried (MgSO₄) and concentrated to dryness. The residue was redissolved in ethyl acetate/hexane 1:1 and purified by filtration through a plug of silica gel eluting with ethyl acetate/hexane 1:1 to give the subtitle compound (0.1 g).

Step 3: (R)-N-Hydroxy-2-(naphthalen-2-ylmethanesulfonylamino)-2-phenyl-acetamide—(R)-(Naphthalen-2-ylmethanesulfoylamino)-N-t-butyldimethylsilyloxy-2-phenyl acetic acid (0.1 g) in THF (0.3 ml) was treated with TBAF(0.3 ml; 1M solution in THF). After 300 min the mixture was diluted with methanol and passed through an SCX column eluting with methanol. Methanol fractions were combined, absorbed onto silica gel (0.2 g) and purified by chromatography (Sep-Pak silica gel cartridge, step gradient 0-10% methanol/DCM) to give the title compound as a yellow solid (15 mg). MS electrospray (−ve ion) 370 (M−H⁻), ¹H NMR δ (CD₃OD) 7.63-7.8(3H,m), 7.58(1H d), 7.28-7.44(3H,m), 7.15-7.3(5H,m), 4.32 and 4.27(2H,AB, J 14 Hz)

### Example 3

### (R)-N-Hydroxy-2-(napthalen-2-ylmethanesulfonylamino)-4-phenyl-butyramide

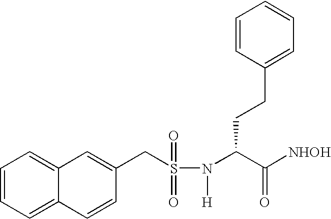

Step 1: (R)-2-(Napthalen-2-ylmethaneslfonylamino)₄-phenylbutyric acid

To (R)phenyl-butyric acid (0.37 g) was added DMF (1 ml), pyridine (1 ml) followed by BSTFA (1.1 ml). The mixture was warmed to 60° C. After 30 min the solution was cooled to 0° C. and a solution of naphthalene-2-yl-methanesulfonyl chloride (0.5 g) in DMF (1 ml) was added dropwise followed by NEt₃ (0.29 ml). The reaction mixture was left at rt for 2 h and then partitioned between ethyl acetate and 1N HCl. The organic layer was concentrated and the residue was purified by chromatography (Sep-Pak silica gel cartridge, step gradient 0-10% methanol/DCM) to afford the subtitle compound (0.34 g).

Step 2: (R)-(Naphthalen-2-ylmethanesulfonylamino)-N-(t-butyldimethylsilyloxy)-4-phenyl-butyramide—A solution of (R)-2-(napthalen-2-ylmethanesulfonylamino)phenyl-butyric acid (0.34 g) in dichloromethane (5 ml) was cooled to 0° C. under argon and treated with 0 t-butyldimethylsilylhydroxylamine (0.196 g) in dichloromethane (1 ml) and EDC-methiodide (0.396 g) in dichloromethane (1 ml). After 2 h at rt the solution was evaporated to dryness. The residue was taken up into ethyl acetate and washed with aqueous sodium bicarbonate. The organic layer was washed with brine, dried (MgSO₄) and concentrated to dryness. The residue was purified by flash chromatography (silica gel, 1:1 ethyl acetate/hexane) to give the subtitle compound (0.32 g).

Step 3: (R)-N-Hydroxy-2-(napthalen-2-ylmethanesulonylamino)-4-phenyl-butyramide—(R)-N-(t-butyldimethylsilyloxy)-2-napthalen-2-ylmethanesulfonylamino)-4-phenyl-butyramide (0.32 g) in dry THF (2 ml) was treated with TBAF (0.6 ml; 1M solution in THF). After 30 min the solution was evaporated to dryness and the residue was partitioned between ethyl acetate and aqueous citric acid (10%). The organic layer was Washed with brine and evaporated to dryness. The residue was purified by chromatography (Sep-Pak silica gel cartridge, step gradient 0-10% methanol/DCM) to give the title compound (0.1 g). MS electrospray (−ve ion) 396.9 (M−H⁻). ¹H NMR δ(DMSO-d₆): 10.8 (1H,s), 9.03 (1H,s,J 1.6 Hz),7.91 (2H,m),7.65(1H,dT 7.65 Hz), 7.53(2H,m), 7.26(2H,m), 7.19(1H,d,J 12 Hz), 7.14(2H,m), 4.47 and 4.41 (2H,ABq,), 3.73(1H ddd J 7,7,7 Hz), 2.4-2.5(2H,m), 1.82(2H,m).

The compounds of the following examples were prepared by the procedures described in Example 2 and Example 3. ¹H NMR spectra and mass spectra were consistent with the structures given in the table.

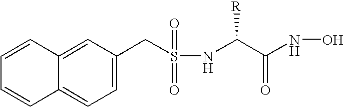

| Example No | 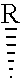 |
|---|---|
| 4 | 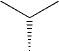 |
| 5 | 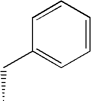 |
| 6 | 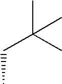 |
| 7 | 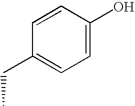 |
| 8 | 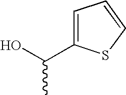 |
| 9 | 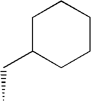 |
| 10 | 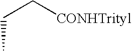 |
| 11 | 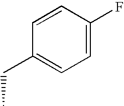 |
| 12 | 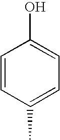 |
| 13 | 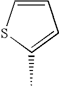 |
| 14 | 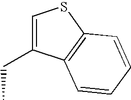 |
| 15 | 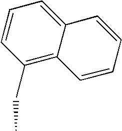 |
| 16 | 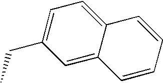 |
| 17 | 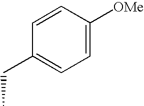 |
| 18 | 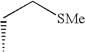 |
| 19 | 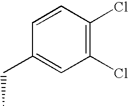 |
| 20 | 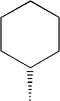 |
| 21 | 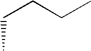 |
| 22 | 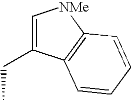 |
| 23 | 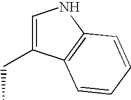 |
| 24 | 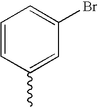 |
| 25 | 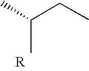 |
| 26 | 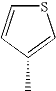 |
| 27 | 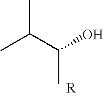 |

N.B. The compounds of Examples 8 and 24 were prepared as racemates.

### Example 28

### (R)-2-(Benzo[b]thiophen-5-ylmethanesulfonylamino)-N-hydroxy-2-phenylacetamide

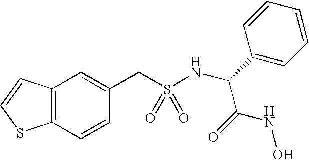

Step 1: R-2-(Benzo[b]thiophen-5-ylmethanesulfonylamino-2-phenylacetic acid—A suspension of (R)-phenylglycine (0.38 g) in pyridine (5 ml) and DMF (5 in) at 55° C. was treated with BSTFA (135 ml). After 30 min at 55° C. the solution was cooled to 0° C. and a solution of benzo[b]thiophene-5-methanesulfonyl chloride (0.75 g) in DMF (2 ml) was added. After a further 2 h at room temperature, ethyl acetate and aqueous potassium hydrogen sulfate were added, and the organic layer was dried (MgSO₄) and evaporated. Crystallisation of the residue from ether gave the subtitle compound (0.40 g).

Step 2: (R)-2-(Benzo[b]thiophen-5-ylmethanesulfonylamino)-N-(t-butyldimethylsilyloxy)-2-phenylacetamide—A solution of (R)-2-(benzo[b]thiophen-5-ylmethanesulfonylamino)-2-phenylacetic acid (0.40 g) in dichloromethane (5 ml) was treated with O-t-butyldimethylsilylhydroxylamine (0.18 g) and EDC-methiodide (0.36 g) at 0° C. After 2 h at rt the solution was treated with ethyl acetate and aqueous sodium hydrogen carbonate. The organic layer was dried (MgSO₄) and evaporated and the residue purified by chromatography (silica gel, ethyl acetate/hexane) to give the subtitle compound (0.37 g).

Step 3: R-2-(Benzo[b]thiophen-5-ylmethanesulfonylamino)-N-hydroxy-2-phenylacetamide—A solution of (R)-2-(benzo[b]thiophen-5-ylmethanesulfonylamino)-N-t-butyldimethylsilyloxy)-2-phenylacetamide (035 g) in THF (5 ml) was treated with TBAF (1 ml, 1M solution in THF). After 30 min the solution was diluted with ethyl acetate and washed with water, aqueous sodium hydrogen carbonate, and brine, dried (MgSO₄) and evaporated. The residue was dissolved in methanol and passed through an SCX cation exchange column eluting with more methanol. Evaporation of the eluant and crystallisation from ether gave the title compound (0.17 g). MS electrospray (−ve ion) 374 (M−H⁻, 100%). ¹H NMR δ(DMSO-d6) 11.0 (1H, bs), 9.0. (1H, bs), 8.13 (1H, d, J=8 Hz), 7.91 (1H, d, J=8 Hz), 7.77 (1H, d, J=6 Hz), 7.70 (1H, s), 7.2-7.4 (8H, m), 4.85 (1H, d, J=9 Hz), 4.28 (2H, ABq).

### Example 29

### (R)-2-Phenyl-2-[(R)-1-(1,2,3,4-tetrahydronaphthalen-2-yl)methanesulfonylamino]-N-hydroxyacetamide

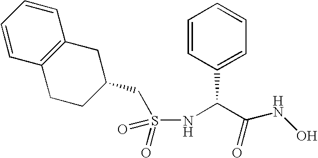

Title compound was prepared from R-1,2,3,4-tetrahydronaphthalene-2-yl)methanesulfonyl chloride using the method described for Example 28 (yield 43%). MS electrospray (−ve ion) 747 (2M−H⁻, 45%), 373 (M−H⁻, 100%).

### Example 30

### (R)-2-(Indan-2-ylmethanesulfonylamino)-2-phenyl-N-hydroxyacetamide

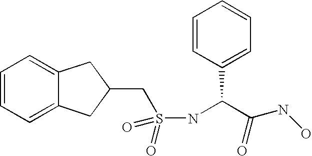

Title compound was prepared from indane-2-methanesulfonyl chloride using the method described for Example 28.

### Example 31

### (R)-2-(Benzo[b]thiophen-5-ylmethanesulfonylamino)-N-hydroxy-3-(1H-indol-3-yl) propionamide.

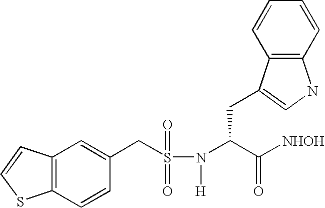

Step 1: (R)-2-(Benzo[b]thiophen-5-ylmethanesulfonylamino)-3-(1H-indol-3-yl)-propionic acid—(R)-Tryptophan (0.3 g) was taken up in DMF (1 ml) and pyridine (1 ml) and treated with BSTFA (0.85 ml). The mixture was warmed to 60° C. After 30 min at 60° C. the solution was cooled to 0° C. and a solution of benzo[b]thiophene-5-methanesulfonyl chloride (0.3 g) in DMF (1 ml) was added dropwise. The reaction mixture was left stirring at rt for 2 h and then treated with methanol (1 ml). The solution was then passed through a Bond Elut PSA column. After initial elution with methanol (30 ml) the product was eluted with 4% TFA in THF (20 ml). The THF solution was stripped to dryness and triturated with hexane to give the subtitle compound (0.3 g).

Step 2: (R)-2-(Benzo[b]thiophen-5-ylmethanesulfonylamino)-N-hydroxy-3-(1H-indol-3-yl)propionnmide—(R)-2-(Benzo[b]thiophen-5-ylmethanesulfonylamino)-3-(1H-indol-3-yl)-propionic acid (0.17 g) was taken up in DMF (4 ml). Solid HOAT (0.06 g) and EDC (0.168 g) were added and the mixture was left stung at rt for 10 min. In a separate flask hydroxylamine hydrochloride (0.091 g) was stirred in DMF (3 ml) followed by the addition of N-methyl morpholine (0.145 ml). The activated propionic acid was then added dropwise to the hydroxylamine solution and allowed to stir at rt for 2 h. After removal of DMF by evaporation the residue was partitioned between citric acid (10%) and ethyl acetate. The organic layer was washed with sodium bicarbonate and brine, then dried (MgSO₄) and concentrated Trituration with ether afforded the title compound as a solid (0.052 g). MS electrospray (−ve ion) 427.8 (M−H⁻); MS electrospray (+ve ion) 429.9 (M+H⁺). ¹H NMR δ(DMSO-d6) 10.86(1H,s), 10.79(1H,s), 7.82(1H,dJ 8 Hz), 7.76(1H,d,J 5.48), 7.66(1H,s), 7.64(1H,d, J=2.3 Hz), 158(1H d, J 7.76), 7.41(1H,d,J 5.28 Hz), 7.33(1H,d,J 7.92 Hz), 7.15-7.01(4H,m), 4.10(1H,d J 13.72 Hz), 4.00(1H,m), 3.89(1H,d J 13.72 Hz), 3.02(1H,dd), 2.85(1H,dd).

The compounds of the following examples were prepared by the process described in Example 31. ¹H NMR spectra and mass spectra were consistent with the structures given in the table.

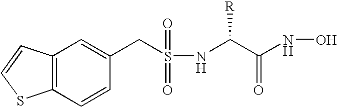

| Example No | 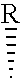 |
|---|---|
| 32 | 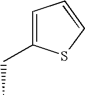 |
| 33 | 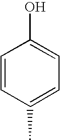 |
| 34 | 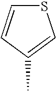 |
| 35 | 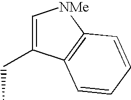 |
| 36 | 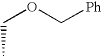 |
| 37 | 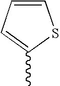 |

N.B. The compound of Example 37 was prepared as a racemate.

### Example 38

### (R)-2-(Benzo[b]thiophen-5-ylmethanesulfonylamino)-N-hydroxy-4-phenyl-butyramide

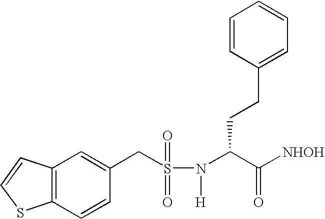

Step 1: (R)-2-(Benzo[b]thiophen-5-ylmethanesulfonylamino)-4-phenyl-butyric acid. (R)-2-(Benzo[b]thiophen-5-ylmethanesulfonylamino)-4-phenyl-butyric acid was prepared according to the method of Example 31 step 1.

Step 2: (R (Benzo[b]thiophen-5-ylmethanesulfonylamino)-N-hydroxy-4-phenylbutyramide. (R)-2-(Benzo[b]thiophene-5-ylmethanesulfonylamino)-4 phenyl-butyric acid (0.1 g) was suspended in DCM (15 ml) and stirred at 0° C. Oxalyl chloride (0.0245 ml) was added, followed by DMF (0.0199 ml) and the reaction mixture stirred at 0° C. for 30 min. In a separate flask, hydroxylamine hydrochloride (0.0709 g) in a mixture of THF (4 ml) and water (1 ml) was stirred at 0° C. Diethylamino polystyrene resin (1.2 g) was added to the stirring hydroxylamine hydrochloride and stirred at 0° C. for 20 min. The acid chloride solution was then added to the hydroxylamine solution, dropwise, at 0° C. and the resultant mixture was then stirred at room temperature for 15 hours. The reaction mixture was then filtered and the filtrate treated with diethylamino polystyrene resin (12 g) and again filtered. The filtrate was treated with SCX cation exchange support (2 g). The SCX was filtered off, and the filtrate was then evaporated and azeotroped with toluene to give the title compound as a pale cream coloured solid (61.5 mg). MS electrospray (−ve ion) 402.6 (M−H. ¹H NMR, δ(DMSO-d6) 8.00(1H, m), 7.96 (1H,s), 7.90(1H,m), 7.78(1H,d,J 5.6 Hz), 7.70(1H,m), 7.61(1H,m),7.48(1H,d,J 5.6), 7.38(1H,dd,J 1.6,4.8), 7.29(2H,m), 7.16(3H,m), 4.38(2H,Abq), 3.71(1H, m), 1.80(2H,m), 0.87(2H, m).

The compounds of the following examples were prepared by the process described in Example 38. ¹H NMR spectra and mass spectra were consistent with the structures given in the table.

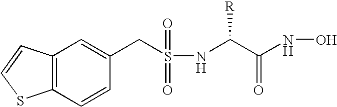

| Example No | 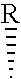 |
|---|---|
| 39 | 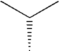 |
| 40 | 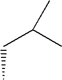 |
| 41 | 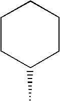 |
| 42 | 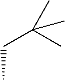 |
| 43 | 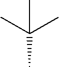 |

### Example 44

### R-2-(Benzo[b]thiophen-6-ylmethanesulfonylamino)-N-hydroxy-2-phenylacetamide

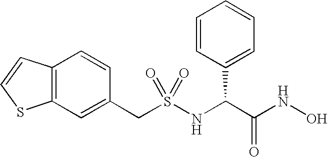

Title compound was prepared using the method described in Example 31. Preparation of the intermediate carboxylic acid was achieved using the method of Example 28 (step 1).

### Example 45

### (R-2-(Benzo[b]thiophen-5-ylmethanesulfonylamino)-3-(1,3-dioxo-1,3-dihydro-isoindol-2-yl)-N-hydroxy propionamide

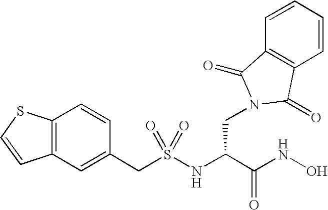

Step 1: (R)-2-Tritylamino-3-(1,3-dioxo-1,3-dihydro-isoindol-2-yl) propionic acid t-butyl ester. (R)-2-Tritylamino-3-hydroxypropionic acid t-butyl ester (0.4 g) in dry THF (10 ml), under argon, was treated with triphenylphosphine (0.26 g) followed by diethyl azodicarboxylate (0.16 ml) dropwise. The solution was stirred at rt overnight and then concentrated to dryness. The residue was taken up into ether (20 ml) and washed with water (3×20 ml) then brine (20 ml). The organic layer was dried (Na₂SO₄) and concentrated. Purification by chromatography (silica gel, step gradient 0-20% ethyl acetate/hexane) afforded the subtitle compound as a white foam (0.36 g).

Step 2. (R)-2-amino-3-(1,3-dioxo-1,3-dihydro-isoindol-2-yl) propionic acid t-butyl ester. (R)-2-Tritylamino-3-1,3-dioxo-1,3-dihydro-isoindol-2-yl) propionic acid t-butyl ester (036 g) in DCM (20 ml) containing triisopropylsilane (0.2 ml) was treated dropwise with TFA (0.4 ml). After 30 min the solution was concentrated to dryness. Trituration with pentane/ether afforded the subtitle compound trifluoroacetate salt (0.24 g). The trifluoroacetate salt in dry dioxan (20 ml) was treated with HCl (20 ml of a 4N solution in dioxan) and the resulting suspension was stirred at rt for 5 h. The mixture was concentrated and then azeotroped with successive portions of toluene. Trituration with ether afforded the sub title hydrochloride salt (0.18 g).

Step 3: (R)-2-(Benzo[b]thiophen-5-ylmethanesulfonylamino)-3-(1,3-dioxo-1 dihydro-isoindol-2-yl) propionic acid t-butyl ester. (R)-2-amino-3-1,3-dioxo-1,3-dihydro-isoindol-2-yl) propionic acid t-butyl ester hydrochloride (0.11 g) in DCM (8 ml) was treated portionwise with naphthalen-2-yl-methanesulfonyl chloride (0.091 g). Pyridine (2 ml) was added dropwise and the mixture was stirred at rt for 5 h. The reaction was concentrated to dryness and then azeotroped with successive portions of toluene. The residue was partitioned between ethyl acetate (10 ml) and water (10 ml). The organic layer was washed with water (2×10 ml), brine (10 ml), dried (Na₂SO₄) and concentrated. The residue was purified by chromatography (silica gel, step gradient 0-5% ethanol/DCM). Trituration with ether afforded the subtitle compound as a white solid. (0.064 g).

Step 4: R)₂-(Benzo[b]thiophen-5-ylmethanesulfonylamino)-3-(1,3-dioxo-1,3-dihydro-isoindol-2-yl)propionic acid. (R)-2-(Benzo[b]thiophen-5-ylmethanesulfonylamino)-3-(1,3-dioxo-1,3-dihydro-isoindol-2-yl) propionic acid t-butyl ester (0.064 g) was treated at ice temperature with TFA (95%) and kept below 10° C. for 3 h. The solution was concentrated to dryness and then azeotroped with successive portions of toluene to give the sub title compound as a white solid (0.058 g).

Step 5: (R)-2-(Benzo[b]thiophen-5-ylmethanesulfonylamino)-3-(1,3-dioxo-1,3-dihydro-isoindol-2-yl)-N-hydroxypropionamide. The title compound was prepared using the method described in Example 31. MS electrospray (−ve ion) 458 (M−H⁻). ¹H NMR δ(DMSO-d6): 10.93 (1H,brs), 9.06 (1H,brs), 7.98 (1H,d,J 5.2 Hz), 7.85 (5H,m), 7.78 (1H,d, J 5.6), 7.45 (1H,d, J 5.2), 7.29 (1H,dd,J 1,6,8.4), 4.40 (2H,ABq), 4.12 (1H, t, J 7.2), 3.71-3.85 (2H,m).

The compounds of the following examples were prepared by the process described in Example 31. ¹H NMR spectra and mass spectra were consistent with the structures.

| | |
|---|---|
| Example 46 | 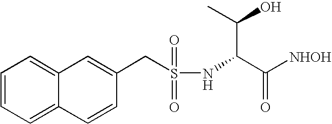 |
| Example 47 | 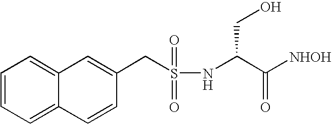 |

### Example 48

### (R)-3-Benzo[b]thiophen-3-yl-2-(benzo[b]thiophene-5-ylmethanesulfonylamino)-N-hydroxy-propionamide

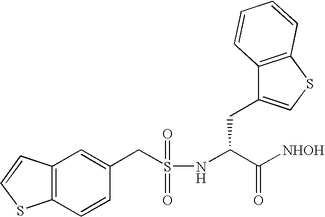

Step 1: (R)-3-Benzo[b]thiophen-3-yl-(benzo[b]thiophen-5-ylmethaneslfonylamino)-propionic acid—Prepared from (R)-2-amino-3-benzo[b]thiophen-3-yl-propionic acid using the method of Example 31 Step 1. Purification of the crude product by solid phase extraction on a Bond Elut NH₂ column afforded the subtitle compound (yield 54%). MS electrospray (−ve ion) 430 (M−H⁻).

Step 2: (R)-3-(Benzo[b]thiophen-3-yl-[b]thiophen-5-ylmethanesulfonylamino)-N-hydroxy-propionamide—Prepared using the method of Example 31 Step 2. After completion of the reaction DMF was removed and the residue was partitioned between aqueous sodium hydrogen carbonate and ethyl acetate. The organic layer was dried (MgSO₄) and concentrated. Purification of the crude product by chromatography (silica gel, step gradient 0-100% ethyl acetate/hexane) afforded the tide compound as a cream solid (27 mg). MS electrospray 444.7(M−H⁻). ¹H NMR δ (DMSO-d₆) 10.91 (1H,br s), 8.99 (1H,s), 7.98 (1H,d, J=8 Hz), 7.90 (1H,d, J=8.4 Hz). 7.84 (1H,d, J=7.6 Hz), 7.77 (1H,d, J 5H), 7.72 (1H,s), 7.42 (4H,m), 7.18 (1H,d, J=8.4 Hz), 4.12 (2H, ABq), 4.09 (14 J 7.6), 3.21 (1H,m), 3.18 (1H,m).

### Example 49

### (2R,3R)-2-(Benzo[b]thiophen-5-ylmethanesulfonylamino)-3-methyl-pentanoic acid—N-hydroxyamide

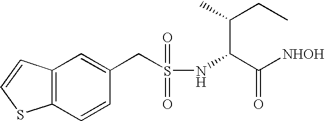

Step 1: (2R,3R)-2-(Benzo[b]thiophen-5-ylmethanesulfonylamino)-3-methyl-pentanoic acid—Prepared using the method of Example 48 Step 1 (yield 64%). MS electrospray(−ve ion) 339.9 (M−H⁻).

Step 2: (2R,3R)-2-(Benzo[b]thiophen-5-ylmethanesulfonylamino)-3-methyl-pentanoic acid-N-hydroxyamide—Prepared using the method of Example 31 Step 2. After completion of the reaction DMF was removed by evaporation and saturated aqueous NaHCO₃ was added. The resulting precipitate was filtered and dried to give the title compound as cream solid (0.14 g). MS electrospray 354.8 (M−H⁻). ¹H NMR δ (DMSO-d₆) 10.81 (1H,s), 9.02 (1H,s), 7.98 (1H,d, J=8.4 Hz), 7.86 (1H,s), 7.78 (1H,d, J=5.6 Hz), 7.46 (2H,d, J=5.6 Hz), 735 (1H,dd, J=82 Hz, 1.6 Hz), 4.31 (2H, ABq), 3.46 (1H,t, J=8 Hz), 1.62 (1H,m), 1.53 (1H,m), 1.09 (1H,m), 0.80 (6H,m).

### Example 50

### (2R,3S)-2-(Benzo[b]thiophen-5-ylmethanesulfonylamino-3-hydroxy-4-methyl-pentanoic acid N-hydroxamide

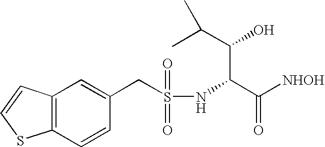

The title compound was prepared from (2R,3S)-2-amino-3-hydroxymethyl-pentanoic acid using the method of Example 48 (cream coloured solid, 30 mg). MS electrospray(+ve ion) 372.9 (M+H⁺). ¹H NMR δ (DMSO-d₆) 10.78 (1H,br s), 8.94 (1H,br s), 7.99 (1H,d, J=8 Hz), 7.90 (1H,s), 7.78 (H,d, J=5.6 Hz), 7.47 (1H,d, J=5.6 Hz), 7.38 (1H,d, J=8.4 Hz), 4.81 (1H,br s), 4.41 (2H, ABq), 3.80 (1H,d, J=5.2 Hz), 1.64 (1H,m), 0.89 (6H,m).

### Example 51

### (R)-2-(Benzo[b]thiophen-5-ylmethanesulfonylamino)-3-tert-butoxy-N-hydroxy-propionamide

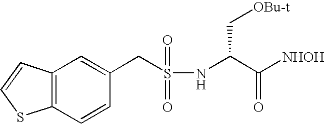

Step 1: (R)₂-(Benzo[b]thiophen-5-ylmethanesulfonylamino)-3-tert-butoxy-propionic acid. Prepared from (R)-2-amino-3-tert-butoxy-propionic acid on a Myriad PS using the method described for Example 31 Step 1 (yield 52%). MS electrospray (−ve ion) 370.0 (M−H⁺).

Step 2: (R)-2-(Benzo[b]thiophen-5-ylmethanesulfonylamino)-3-tert-butoxy-N-hydroxy-propionamide—Prepared from (R)-2-(Benzo[b]thiophen-5-ylmethanesulfonylamino)-3-tert-butoxy-propionic acid using the method of Example 38 Step 2. The reaction mixture was treated with diethylamino polystyrene resin then filtered, and the filtrate was evaporated to dryness. The residue was purified on a Biotage Parallex HPLC to give the title compound as a white solid (6 mg). MS electrospray (−ve ion) 385.1(M−H⁻). ¹H NMR 8 (DMSO-d₆) 10.71 (1H,s), 9.00 (1H,s), 7.99 (1H,d, J=8.3 Hz), 7.87 (1H,s), 7.79 (1H,d, J=5.4 Hz), 7.47 (2H,m), 7.36 (1H,dd, J=8.3 Hz, 1.48 Hz), 4.43 (2H,s), 3.79 (1H,m), 3.43 (1H,m), 3.29 (1H,m), 1.09 (9H,s).

### Example 52

### (R)-2-(Benzo[b]thiophen-5-ylmethanesulfonylamino-N-hydroxy-4-morpholin-4-yl-4-oxo-butyramide

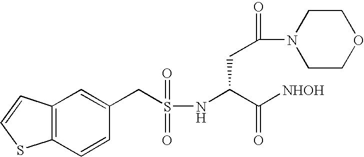

Step 1: (R)-2-tert-Butoxycarbonylamino-4-morpholn-yl-4-oxo-butyric acid tert butyl ester—To a solution (R)-2-tert-butoxycarbonylamino-succinic acid 1-tert-butyl ester (0.5 g) in DCM (15 ml) were added HOAT (0.55 g), EDC (0.73 g) and morpholine (0.149 g), and the reaction allowed to stir at rt for 2 h. Solvent was removed by evaporation and the oily residue was partitioned between aqueous sodium bicarbonate and ethyl acetate. The biphasic mixture was passed through a hydromatrix stationary phase which retained the aqueous layer. The organic layer was concentrated to give the subtitle compound as a white solid (0.55 g).

Step 2: (2-Amino-4-morpholin-4-yl-4-oxo-butyric acid hydrochloride—(R)-2-tert-Butoxycarbonylamino-4-morpholin-4-yl-4-oxo-butyric acid tert-butyl ester (0.5 g) was dissolved in dry DCM (5 ml) followed by the addition of 4N HCl in dioxan (4 ml). The reaction was allowed to stir at rt overnight. The solvent was removed by evaporation to give the subtitle compound as a white solid (0.3 g).

Step 3: (R)-2-(Benzo[b]thiophen-5-ylmethanesulfonylamino)-4-morpholin-4-oxo-butyric acid—Prepared using the method of Example 48 step 1 (yield 34%). MS electrospray (+ve ion) 412.8 (M+H⁺).

Step 4: (R)-2-(Benzo[b]thiophen-5-ylmethanesulfonylamino)-N-hydroxy-4-morpholin-4-yl-4-oxo-butyramide—Prepared using the method of Example 31 Step 2. The title compound was obtained as a white solid after tritration with hexanel ether (yield 39%). MS electrospray (+ve ion) 427.8 (M+H⁺). ¹H NMR δ (DMSO-d₆) 10.79 (1H,s), 8.97 (1H,s), 7.98 (1H,d, J=8 Hz), 7.86 (1H,s), 7.79 (1H,d, J=5.6 Hz), 7.46 (2H,d, J=5.6 Hz), 7.35 (1H,d, J=8 Hz), 4.46 (2H,s), 4.20 (1H,d, J=6.8 Hz), 354 (4H,m), 3.44 (2H,m), 3.40 (2H,m), 2.67 (2H,m)

### Example 53

### (R)-2-(Benzo[b]thiophen-5-ylmethanesulfonylamino)-S-morpholin-4-yl-5-oxo-pentanoic acid N-hydroxyamide

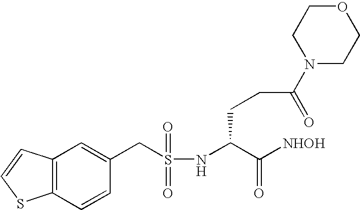

Step 1: (R)-2-(Benzo[b]thiophen-5-ylmethanesulfonylamino)pentanedioic acid-1-tert-butyl ester—Prepared from (R)-2-amino-pentanedioic acid 1-tert-butyl ester using the method described for Example 48 step 1 (yield 15%). MS electrospray (−ve ion) 411.9 (M−H⁻).

Step 2: (R)-2-(Benzo[b]thiophen-5-ylmethanesulfonylamino)-5-morpholin-4-yl-5-oxo-pentanoic acid-tert-butyl ester—A solution of (R)-2(benzo[b]thiophen-5-ylmethanesulfonylamino)pentanedioic acid-1-tert-butyl ester (0.3 g) in DMF (3 ml) was treated with with HOAT (0.11 g), EDC (0.278 g) and morpholine (0.063 g). The reaction was stirred at rt for 2 h. Solvent was was removed by evaporation and the residue partitioned between aqueous sodium bicarbonate and ethyl acetate. The organic layer was dried (MgSO₄) and concentrated. The residue solidified on standing to give the subtitle compound (yield 100%). MS electrospray (−ve ion) 480.9 (M−H⁻).

Step 3: (R)-2-Benzo[b]thiophen-5-ylmethanesulfonylamino)-5-morpholin-4-yl-5-oxo pentanoic acid—Prepared from (R)-2-(benzo[b]thiophen-5-ylmethanesulfonylamino)-5-morpholin-4-yl-5-oxo pentanoic acid-tert-butyl ester using the method described for Example 52 Step 2 (yield 89%). MS electrospray (−ve ion) 424.9 (M−H⁻).

Step 4: (R)-2-(Benzo[b]thiophen-5-ylmethanesulfonylamino)-5-morpholin-4-yl-5-oxo-pentanoic acid N-hydroxyamide—Prepared from (R-2-(Benzo[b]thiophen-5-ylmethanesulfonylamino)-5-morpholin-4-yl-5-oxo pentanoic acid using the method described for Example 31 Step 2. After work-p the crude reaction mixture was purified on a Biotage Parallex HPLC to give the title compound as a white solid (20 mg). MS electrospray(+ve ion) 441.9 (M+H⁺), MS electospray (−ve ion) 439.9(M−H⁻). ¹H NMR δ (DMSO-d₆) 10.77 (s, 1H), 8.99 (1H,br s), 7.98 (1H,d, J=8.4 Hz), 7.88 (1H,s), 7.78 (1H,d, J=5.6 Hz), 7.52 (1H,d, J=8.8 Hz), 7.46 (1H,d, J 5.6 Hz), 7.37 (1H,dd, J=8.4 Hz, 1.6 Hz), 4.39 (2H,s), 3.72 (1H,dd, J=15.2 Hz, 7.2 Hz), 354 (4H,m), 2.31 (4H,m), 1.81 (4H,m).

### Example 54

### (R)-2-(Benzo[b]thiophen-5-ylmethanesulfonylamino-2-(4-fluorophenyl)-N-hydroxy-acetamide

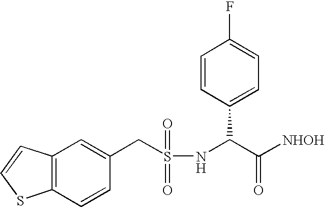

Step 1: (R)-2-(Benzo[b]thiophen-5-ylmethanesulfonylamino)-2-(4-fluoro-phenyl)-acetic acid—Prepared from (R)-4-fluorophenylglycine using the method in Example 31 Step 1 (95% yield). MS electrospray (−ve ion) 477.9(M−H⁻).

Step 2: (R)-2-(Benzo[b]thiophen-5-ylmethanesulfonylamino)-2-(4-fluoro-phenyl)-N-hydroxy-acetamide—Prepared on a Myriad PS using the method described for Example 38 Step 2. The crude reaction mixture was purified on a Biotage Parallex HPLC to give the title compound (15.7 mg). MS electrospray (−ve ion) 392.9(M−H⁻). ¹H NMR δ (DMSO-d₆) 10.91 (1H,br s), 9.02 (1H,br s), 7.93 (1H,d, J=8 Hz), 7.77 (1H,d, J=5.6 Hz), 7.71 (1H,s), 7.42 (4H,m), 7.26 (1H,dd, J=8.2 Hz, 1.6 Hz), 7.15 (1H,t, J=8.8 Hz), 4.83 (1H,s), 4.41 (2H, ABq). Chiral purity: ee>99%.

### Example 55

### (R,S)-2-(Benzo[b]thiophen-5-ylmethanesulfonylamino)-2-(3-chloro-4-fluorophenyl)-N-hydroxy-acetamide—Prepared from (R,S)-3-chloro-4-fluorophenylglycine using the method of Example 54.

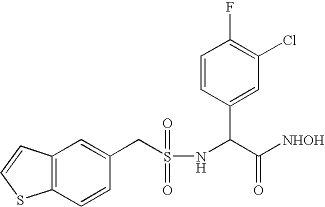

### Example 56

### (R)-2-(Benzo[b]thiophen-5-ylmethanesulfonylamino)-2-(4-isopropylphenyl)-N-hydroxy-acetamide

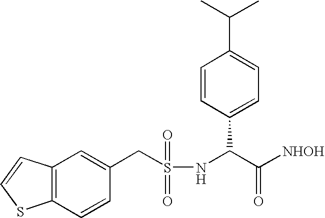

Step 1: (R,S)-(enzo[b]thiophen-5-ylmethanesulfonylamino)-2-(4-isopropyl-phenyl)-acetic acid—Prepared from 4-isopropylphenylglycine using the method of Example 31 Step 1 (0.41 g). MS electrospray (−ve ion) 401.9 (M−H⁻).

Step 2: (R)-2-(Benzo[b]thiophen-5-ylmethanesulfonylamino)-2-(4-isopropyl-phenyl)-N-hydroxy-acetamide—Prepared in a similar manner to Example 38 Step 2. The crude racemic product was separated into single enantiomers using chiral preparative HPLC. Fractions containing the slower running component afforded the title compound as a white solid (42 mg). MS electrospray (−ve ion) 416.9 (M−H); Chiral purity: ee>99.9%; [α]_{D}²²=−47° (c 0.1, MeOH); ¹H NMR δ (DMSO-d₆) 10.90 (1H,s), 9.03 (1H,s), 8.04 (1H,br s), 7.93 (1H,d, J=8.4 Hz), 7.77 (1H,d, J=5.6 Hz), 7.70 (1H,s), 7.39 (1H,d, 14.8 Hz), 7.33 (2H,d, J=8.4 Hz), 7.24 (1H,dd, J=8.4 Hz, 1.6 Hz), 7.20 (2H,d, J=8.4 Hz) 4.81 (1H,s), 4.30 (2H, ABq), 2.87 (1H,m), 1.19 (6H,d, J=60.5 Hz).

### Example 57

### (R)-2-(Benzo[b]thiophen-5-ylmethanesulfonylamino)-2-(3-fluorophenyl)-N-hydroxy-acetamide

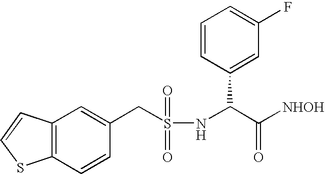

Step 1: (R,S)-(Benzo[b]thiophen-5-ylmethanesulfonylamino)-2-(3-fluoro-phenyl)-acetic acid—Prepared from (R,S)-3-fluorophenylglycine using the method described for Example 31 Step 1 (pale yellow solid, 1.23 g). MS electrospray (−ve ion) 377.9 (M−H⁻).

Step 2: (R)-2-(Benzo[b]thiophen-5-ylmethanesulfonylamino)-2-(3-fluoro-phenyl)-N-hydroxy-acetamide—Prepared on a Myriad PS using a similar method to that described for Example 38 Step 2. The crude racemic product was separated into single enantiomers using chiral preparative HPLC. Fractions containing the slower running component afforded the title compound as a white solid (32 mg). MS electrospray (−ve ion) 392.8 (M−H⁻). Chiral purity: ee>93%, [a]Dm=610 (c 0.1, MeOH). ¹H NMR 5 (DMSO-d₆) 10.99 (1H,s), 9.11 (1H,s), 8.23 (1H,d, J=9.2 Hz), 7.93 (1H,d, J=8.4 Hz), 7.77 (1H,d, J=5.2 Hz), 7.72 (1H,s), 7.38 (2H,m), 7.23 (3H,m), 7.12 (1H,m), 4.86 (1H,d, J=9.2 Hz), 4.34 (2H, ABq).

### Example 58

### (R)-2-(Benzo[b]thiophen-5-ylmethanesulfonylamino)-2-(4-trifluoromethoxyphenyl)-N-hydroxy-acetamide

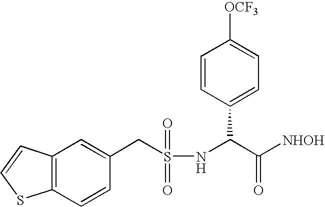

Step 1: (R,S)-N-Formyl-trifluoromethoxyphenylglycine—To a solution of (R,S)-4-trifluoromethoxyphenylglycine (5.0 g) in formic acid (100 ml) was added acetic anhydride (20 ml). After 18 h at 55° C. evaporation and crystallisation of the residue from water gave the subtitle compound (3.8 g).

Step 2: (R)-N-Formyl-4-trifluoromethoxyphenylglycine—A solution of (R,S)N-formyl-4-trifluoromethoxyphenylglycine (3.8 g) in 0.1M aqueous phosphate buffer (pH7, 1 litre) was treated with acylase I (grade m from pig kidney, 7000 units/mg, 40 mg). After 14 days at rt the solution was acidified with 2M HCl and extracted with ethyl acetate. The organic layer was dried (MgSO₄), evaporated and the residue crystallised from diethyl ether to give the subtitle compound (2.1 g). [α]_{D}²⁰−144° (c 1, MeOH)

Step 3: (R)-4-Trifluoromethoxyphenylglycine hydrochloride—A mixture of of (R)-N-formyl-4-trifluoromethoxyphenylglycine (11.0 g) and hydrochloric acid (5M, 20 ml) was refluxed for 30 min then evaporated and the residue crystallised from diethyl ether to give the subtitle compound (0.7 g). [a]D; −78° (c 1, 1M aq HCl)

Step 4: (R)-2-(Benzo[b]thiophen-5-ylmethanesulonylamino)-2-(4-trifluoromethoxyphenyl) acetic acid—Prepared on a Myriad PS using the method described in Example 31 step 1 (0.15 g). MS electrospray (−ve ion) 459.0 (M−H⁻).

Step 5: (R)-2-(Benzo[b]thiophen-5-ylmethanesulfonylamino)-2-(4-trifluoromethoxyphenyl)-N-hydroxy-acetamide—Prepared using a similar method to Example 38 Step 2. The crude product was purified by chromatography (SeP-Pak silica gel cartridge, step gradient 0-10% MeOH/DCM). Fractions containing the product were evaporated to dryness, dissolved in methanol (10 ml) and passed through a column packed with diethylaminomethyl polystyrene resin (3 g) to remove traces of hydroxylamine hydrochloride. Concentration of the collected fractions afforded the title compound (10 mg). MS electrospray (−ve ion) 458.9 (M−H⁻). Chiral purity: ee>99%. ¹H NR 8 (DMSO-d₆) 10.94 (1H,br s), 9.09 (1H,s), 7.92 (d, 1H,d, J 8.4 Hz), 7.77 (1H,d, J=5.6 Hz), 7.73 (1H,s), 7.50 (2H,d, J=8.8 Hz), 738 (1H,d, J 5H), 733 (2H,d, J=8.4 Hz) 7.26 (1H,dd, J=8 Hz, 1.6 Hz) 4.88 (1H,s), 4.35 (2H, ABq).

### Example 59

### (R)-2-(Benzo[b]thiophen-5-ylmethanesulfonylamino)-N-hydroxy-2-(4 trifluoromethane-sulfonyloxyphenyl)-acetamide

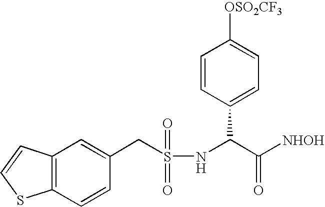

Step 1: (R)-2-Benzo[b]thiophen-5-ylmethanesulfonylamino)-2-(4-hydroxyphenyl) acetic acid-tert-butyl ester—A solution of (R)-2(benzo[b]thiophen-5-ylmethanesulfonylamino)-2-4-hydroxyphenyl)-acetic acid (2.0 g) in dry DCM (15 ml) was treated dropwise with O-tert-butyl-N,N′-diisopropylisourea (0.316 g) over 45 min. After 3 h the reaction mixture was filtered and the filtrate was absorbed onto silica and purified by chromatography (Sep-Pak silica gel cartridge, hexane ethyl acetate, gradient 3% steps of ethyl acetate) to give the subtitle compound (0.6 g). MS electrospray (−ve ion) 431.9 (M−H⁻).

Step 2: (R)-(Benzo[b]thiophen-5-ylmethanesufonylamino)-(4-trifluoromethanesulfonyloxyphenyl)-acetic acid tert-butyl ester—A solution of (R)-2-(benzo[b]thiophen-5-ylmethanesulfonylamino)-2(4-hydroxyphenyl)-acetic acid-tert-butyl ester (0.5 g) in dry MeCN (15 ml) was treated with Hunigs base (0.178 g). The reaction mixture was cooled to 0° C. followed by portion wise addition of N-phenyl-triflimide (05 g) and left stirring overnight. The crude reaction mixture was absorbed onto silica and purified by chromatography (Sep-Pak silica gel cartridge, hexane/ethyl acetate gradient in 3% steps of ethyl acetate). The product eluted with 24% ethyl acetate/hexane and was isolated as a waxy solid (037 g). MS electrospray (−ve ion) 563.75 (M−H⁻).

Step 3: (R)-(Benzo[b]thiophen-5-ylmethanesulfonylamino)-(4-trifluoromethanesulfonyloxyphenyl)-acetic acid—Prepared using the method of Example 52 step 2 (white solid, 0.1 g). MS electrospray (−ve ion) 507.9 (M−H⁻).

Step 4: (R)-2-(Benzo[b]thiophen-5-ylmethanesulfonylamino)-N-hydroxy-2-(4-trifluoromethane-sulfonyloxyphenyl)-acetamide—Prepared using a similar method to Example 38 Step 2. After work-up the crude solid was purified on a Biotage Parallex HPLC to give the title compound as a white solid (6 mg). MS electrospray (−ve ion) 507.9 (M−H⁻). ¹H NMR (DMSO-d₆) 10.99 (1H,s), 9.14 (1H,s), 8.28 (1H,d, J=9.2 Hz), 7.92 (1H,d, J=8 Hz), 7.77 (1H,d, J=5.2 Hz), 7.73 (1H,s), 756 (2H,d, J=8.8 Hz), 7.47 (2H,d, J=8.8 Hz), 7.39 (1H,d, J=5.2 Hz), 7.25 (1H,dd, J=8.4 Hz, 1.6 Hz), 4.90 (1H,d, J=8.8 Hz), 4.37 (2H, ABq).

The compounds of the following examples were prepared by the process described in Example 31. ¹H NMR spectra and mass spectra were consistent with the structures given in the table.

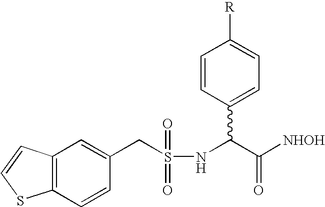

| Example No | R |
|---|---|
| 60 | Br |
| 61 | CF₃ |
| 62 | OMe |
| 63 | Cl |
| 64 | Me |

### Example 65

### (R)-2-(Benzo[b]thiophen-5-ylmethanesulfonylamino)-N-hydroxy-3-(toluene-4-sulfonylamino)-propionamide

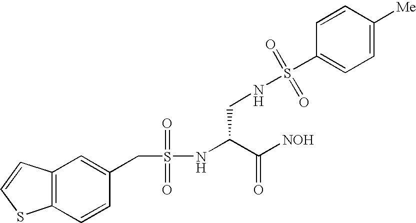

Step 1: (R)-2-tert-Butoxycarbonylamino-3-(toluene-4-sulfonylamino)-propionic acid—To a solution of (R)-2-tert-butoxycarbonyl-3-aminopropionic acid (2.0 g) in 1,4-dioxan (40 ml) and water (20 ml) was added triethylamine (6.9 ml) followed by toluene-4-sulfonyl chloride (2.2 g). The solution was then stirred at rt for 24 h, concentrated, and then partitioned between aqueous sodium hydrogen carbonate and ethyl acetate. The organic layer was dried (MgSO₄) and evaporated to give the subtitle compound (2.4 g).

Step 2: (R)-2-Amino-3-(toluene-4-sulfonylamino)-propionic acid hydrochloride—To a solution of (R)-2-tert-butoxycarbonylamino-3-(toluene-4-sulfonylamino)propionic acid (2.3 g) in dichloromethane (40 ml) was added a 4M solution of hydrogen chloride in 1,4-dioxan (10 ml). After 2 h at rt the mixture was evaporated and the resulting residue crystallised from diethyl ether to give the subtitle compound (1.8 g). ¹H NMR (DMSO-d₆): 8.4(3H,bs), 8.1(1H,bs), 7.4 and 7.7(4H,ABq), 3.9(1H,m), 3.6(3H,s), and 3.2(2H,m).

Step 3: (R)-2-(Benzo[b]thiophen-5-ylmethanesulfonylamino)-3-(toluene-4-sulfonylamino)-propionic acid—To a mixture of (R)-2-amino-3-(toluene-4-sulfonylamino)-propionic acid hydrochloride (0.36 g), pyridine (1 ml), and DMF (1 ml) was added BSTFA (1.3 ml). After 30 min at rt the clear solution was cooled to 0° C. and a solution of benzo[b]thiophene-5-methanesulfonyl chloride (0.9 g) in DMF (2 ml) was added. After stirring for 1 h at rt the reaction mixture was purified by solid phase extraction on a Bond Elut PSA column using the procedure described in Example 31 to give the subtitle compound (0.36 g).

Step 4: (R)-2-(Benzo[b]thiophen-5-ylmethanesulfonylamino)-N-hydroxy-3-(toluene-4-sulfonylamino)-propionamide—Prepared according to the method of Example 31 step 2, yield 55%. Electrospray MS (−ve ion) 482 (M-F, 100%). ¹H NMR δ(DMSO-d₆): 11.0(1H,bs), 9.1(1H,bs), 7.3-8.0(11H,m), 4.4(2H,ABq), 3.9(1H,m), 3.0(1H,m), 2.8(1H,m), 2.3(3H,s).

The compounds of the following examples were prepared by the procedures described in Example 65. ¹H NMR spectra and mass spectra were consistent with the structures given in the table.

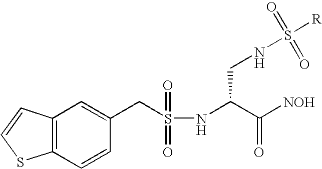

| Example No | R |
|---|---|
| 66 | 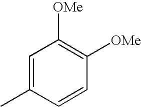 |
| 67 | 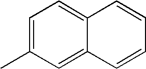 |
| 68 | 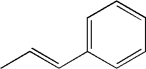 |

### Example 69

### (R)-2-(Benzo[b]thiophen-5-ylmethanesulfonylamino)-N-hydroxy-3-(phenylmethanesulfonylamino)-propionamide

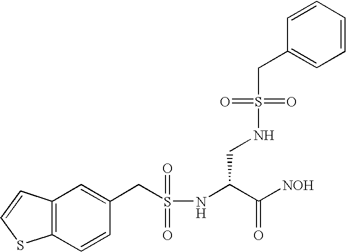

Step 1: (R)-2-tert-Butoxycarbonylamino-3-(phenylmethanesulfonylamino)-propionic acid—A solution of (R)-2-tert-butoxycarbonyl-3-aminopropionic acid (1.0 g) in pyridine (5 ml) and DMF (5 ml) was treated with BSTFA (3.9 ml). After 30 min at rt the reaction was cooled to 0° C. and a solution of phenylmethanesulfonyl chloride (0.9 g) in DMF (2 ml) was added. After stirring for 1 h at rt the reaction mixture was partitioned between 1M aqueous citric acid and ethyl acetate. The organic layer was washed with water and brine, dried (MgSO₄), and evaporated. Chromatography (silica gel, dichloromethane-methanol) gave the subtitle compound (0.5 g).

Step 2: (R)-2-Amino-3-(phenylmethanesulfonylamino)-propionic acid hydrochloride—Prepared by the method of Example 65, step 2.

Step 3: (R)-2-(Benzo[b]thiophen-5-ylmethanesulfonylamino)-3-(phenylmethanesulfonylamino)-propionic acid—Prepared by the method of Example 65 step 3.

Step 4: (R)-2-(Benzo[b]thiophen-5-ylmethanesulfonylamino)-N-hydroxy-3-(phenylmethanesulfonylamino)-propionamide—Prepared by the method of Example 31 step 2, yield 8%. Electrospray MS (−ve ion) 482 (M−H⁻, 100%). ¹H NMR δ(DMSO-d₆): 10.9(1H,bs), 9.1(1H,bs), 7.3-8.0(12H,m), 4.5(2H,s), 4.4(2H,s), 3.9(1H,m), 3.2(1H,m), and 3.0(1H,m).

### Example 70

### (R)-2-(Benzo[b]thiophen-5-ylmethanesulfonylamino)-N-hydroxy-3-pyrazol-1-yl-propionamide

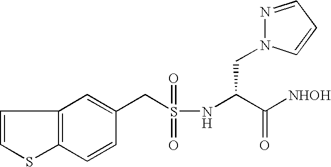

Step 1: (R)-2-tert-Butoxycarbonylamino-3-pyrazol-1-yl-propionic acid—Pyrazole (0.185 g, 2.7 mmol) was taken up in MeCN (9 ml) and added to N-Boc-D-serine P-lactone (Vederas, Arnold, Kalantar, J. Am. Chem. Soc. 1985, 107, 7105) (0.5 g) in MeCN (12 ml) followed by stirring overnight at 50° C. Solvent was removed in vacuo to yield a crude oil (630 mg) that was taken up in methanol and transferred to a Bond Elut PSA column. The column was washed with methanol prior to elution with 4% TFA/THF which afforded the subtitle compound (280 mg).

Step 2: (R)-2-Amino-3-pyrazol-1-yl-propionic acid hydrochloride—A solution of (R)-2-tert-butoxycarbonylamino-3-pyrazol-1-yl-propionic acid (230 mg) in DCM (6 ml) was treated with 4M HCl in dioxan (4 ml) and left to stir for 4 h. Evaporation of solvent afforded the subtitle compound (152 mg).

Step 3: (R)-2-(Benzo[b]thiophen-5-ylmethanesulfonylamino)-3-pyrazol-1-yl-propionic acid—(R)-2-Amino-3-pyrazol-1-yl-propionic acid hydrochloride (84 mg) in DMF (1 ml) and pyridine (1 ml) was treated with BSTFA (0.46 ml) and then stirred at rt for 1 h. The reaction mixture was then cooled to 0° C. and benzo[b]thiophen-5-methanesulfonyl chloride (120 mg) in DMF (1 ml) was added. The reaction mixture was left to stir for 2 h and then quenched with methanol (1 ml). The solution was purified by solid phase extraction on a Bond Elut PSA column, washing with methanol and then eluting with 4% TFA/THF to give the subtitle compound (57 mg).

Step 4: (R)-2-(Benzo[b]thiophen-5-ylmethanesulfonylamino-N-hydroxy-3-pyrazol-1-1-yl-propionamide—(R)-2-(Benzo[b]thiophen-5-ylmethanesulfonylamino)-3-pyrazol-1-yl-propionic acid (100 mg) was taken up in DMF (2 ml), treated with EDC (104 mg) and HOAT (37 mg) and stirred for 5 min. In a separate flask, hydroxylamine hydrochloride (57 mg) in DMF (1 ml) was treated with NMM (0.086 ml) and stirred for 5 min. The solution of activated acid was added dropwise to the hydroxylamine and stirred for 2 h at rt. Solvent was evaporated and aqueous sodium hydrogen carbonate and ethyl acetate were added. The layers were separated and the aqueous phase was washed with ethyl acetate. The organic layers were dried (MgSO₄) and concentrated, and the resulting crude oil was purified on a Biotage Parallex HPLC to give the title compound (14 mg) ¹H NMR (DMSO-d₆) 10.94 (1H,br s), 9.10 (1H,br s), 7.94 (1H,d, J=8 Hz), 7.78 (1H,m), 7.75 (1H,s), 7.65 (1H,m), 751 (1H,s), 7.45 (1H,m) 7.22 (1H,m), 6.25 (1H,m), 4.25 (3H,m), 4.07 (2H, Abq).

The compounds of the following Examples were prepared by the process described in Example 70. ¹H NMR and mass spectra were consistent weith the structures given in the table.

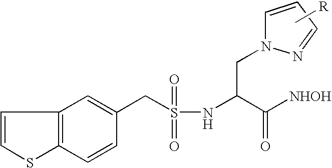

| Example No | R |
|---|---|
| 71 | 3,5-dimethyl |
| 72 | 4-Br |
| 73 | 4-Br-3,5-dimethyl |

### Example 74

### (R)-2-(2-Fluorobenzo[b]thiophen-5-ylmethanesulfonylamino)-N-hydroxy-2-phenylacetamide

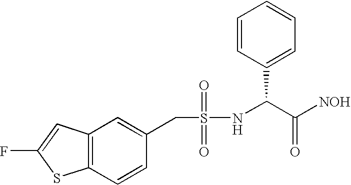

Step 1: (R)-2-(2-Fluorobenzo[b]thiophen-5-ylmethanesulfonylamino)-2-phenylacetic acid—Prepared by the method of Example 65 step 3.

Step 2: (R)-2-(2-Fluorobenzo[b]thiophen-5-ylmethanesulfonylamino)-N-hydroxy-2-phenyl-acetamide—Prepared by the method of Example 38 step 2, yield 38%. Electrospray MS (−ve ion) 393 (M−H⁻, 65%), 229 (100%). ¹H NMR δ(DMSO-d₆): 11.0(1H,bs), 9.0(1H,bs), 8.2(1H,bs), 7.0-7.8(9H,m), 4.9(1H,s), 4.2(2H,ABq).

### Example 75

### (R)-2-(3-Fluorobenzo[b]thiophen-5-ylmethanesulfonylamino)-N-hydroxy-2-phenylacetamide

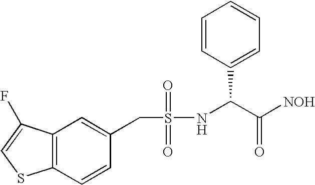

Step 1: (R)-2-(3-Fluorobenzo[b]thiophen-5-ylmethanesulfonylamino)-2-phenylacetic acid—Prepared by the method of Example 65 step 3.

Step 2: (R)-2-(3-Fluorobenzo[b]thiophen-5-ylmethanesulfonylamino)-N-hydroxy-2-phenyl-acetamide—Prepared by the method of Example 38 step 2, yield 15%. Electrospray MS (−ve ion) 393 (M−H⁻, 25%), 165 (100% o). ¹H NMR δ(DMSO-d₆): 11.0(1H,s), 9.0(1H,s), 8.2(1H,d), 7.2-7.9(9H,m), 4.8(1H,d), 4.2(2H,ABq).

### Example 76

### (R)-2-(6-Fluorobenzo[b]thiophen-5-ylmethanesulfonylamino)-N-hydroxy-2-phenylacetamide

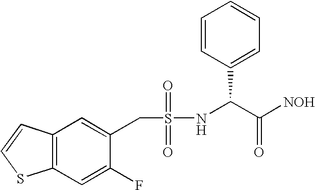

Step 1: (R)-2-(6-Fluorobenzo[b]thiophen-5-ylmethanesulfonylamino)-2-phenylacetic acid—Prepared by the method of Example 65 step 3.

Step 2: (R)-2-(6-Fuorobenzo[b]thiophen-5-ylmethanesulfonylamino)-N-hydroxy-2-phenyl-acetamide—Prepared by the method of Example 38 step 2, yield 17%. Electrospray MS (−ve ion) 393 (M−H⁻, 68%), 165 (100%). ¹H NMR (DMSO-d₆): 11.0(1H,s), 9.0(1H,s), 8.3(1H,d), 7.3-7.9(9H,m), 4.9(1H,m), 4.2(2H,ABq).

Abbreviations

Bond Elut PSA bonded silica supplied by Varian

Bond Elut NH₂ bonded silica supplied by Varian

BSTFA—N,O-Bis trimethylsilyltrifluoroacetamide

DCM—Dichloromethane

Diethylamino Polystyrene resin supplied by FLUKA

DMF—N,N-Dimethylformamide

EDC—1-(3-dimethylaminopropyl)-3-ethylcarbodiimide hydrochloride

EDC—methiodide—1-(3-dimethylaminopropyl)-3-ethylcarbodiimide methiodide

HOAT—1-Hydroxy-7-azabenzotriazole

NMM—N-Methylmorpholine

NMP—1-methyl-2-pyrrolidinone

PS isocyanate—polymer supported isocyanate supplied by Argonaut

SeP-Pak silica gel cartridges supplied by Waters

Technologies

rt—Room temperature

SCX—Bonded silica cation exchange support supplied by Varian

TBAF—Tetra-n-butylammonium fluoride

TFA—Trifluoroacetic acid

THF—Tetrahydrofuran

TMMA—N,N,N′,N′-tetramethylethylenediamine

Trityl—Triphenylmethyl

HPLC Conditions

Analytical and preparative chiral HPLC separations were carried out using ChiralPak AD columns and ethanol/hexane as eluant.

Preparative separations were carried out on a Biotage Parallex HPLC eluting with solvent A (0.1% TFA in water) and solvent B (0.1% TFA in acetonitrile); gradient (10-100% acetonitrile).

Automated synthesis carried out on a Myriad PS supplied by Mettler Toledo.

1. A compound of formula (I): 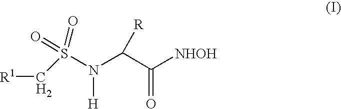
wherein
R is hydrogen, alkyl, alkenyl, alkynyl, aryl, heteroaryl or heterocyclyl; and
R¹ is bicyclyl or heterobicyclyl.

2. A compound of formula (IA): 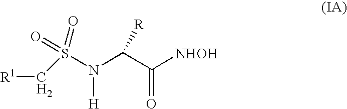
wherein
R is hydrogen, alkyl, alkenyl, alkynyl, aryl, heteroaryl or heterocyclyl; and
R¹ is bicyclyl or heterobicyclyl.

3. A compound according to claim 1 or claim 2 wherein R is isobutyl, phenyl, 4-hydroxyphenyl, 4-fluorophenyl, 4-isopropylphenyl, 3-fluorophenyl, indol-3-ylmethyl, benzothiophen-3-yl, benzothiophen-3-ylmethyl, 4-trifluoromethoxyphenyl, 4-trifluoromethanesulfonyloxyphenyl, phenylmethanesulfonylaminomethyl, phenethyl or pthalimidomethyl and/or R¹ is 2-naphthyl, (R)-1,2,3,4-tetrahydronaphthalen-2-yl, benzothiophen-5-yl optionally substituted by fluorine, benzothiophen-6-yl or indan-2-yl.

4. A compound selected from the group consisting of
(R)-N-Hydroxy-4-Methyl-2-(naphthalen-2-ylmethanesulfonylamino)-pentanoic acid amide
(R)-N-Hydroxy-2-(naphthalen-2-ylmethanesulfonylamino)-2-phenyl-acetamide
(R)-N-Hydroxy-2-(napthalen-2-ylmethanesulfonylamino)-4-phenyl-butyramide
(R)-2-(Benzo[b]thiophen-5-ylmethanesulfonylamino)-N-hydroxy-2-phenylacetamide
(R)-2-Phenyl-2-[(R)-1-(1,2,3,4-tetrahydronaphthalen-2-yl)methanesulfonylamino]-N-hydroxyacetamide
(R)-2-(Indan-2-ylmethanesulfonylamino)-2-phenyl-N-hydroxyacetamide
(R)-2-(Benzo[b]thiophen-5-ylmethanesulfonylamino)-N-hydroxy-3-(1H-indol-3-yl) propionamide
(R)-2-(Benzo[b]thiophen-5-ylmethanesulfonylamio)-N-hydroxyphenyl-butyramide
(R)-2-(Benzo[b]thiophen-ylmethanesulfonylamino)-N-hydroxy-2-phenylacetamide
(R)-2-(Benzo[b]thiophen-5-ylmethanesulfonylamino)-N-hydroxy-2-(4-hydroxyphenyl)acetamide
(R)-2-Benzo[b]thiophen-5-ylmethanesulfonylamino)-3-(1,3-oxo-1,3-dihydro-isoindol-2-yl)-N-hydroxy propionamide
(R)-3-Benzo[b]thiophen-3-yl-2-(benzo[b]thiophene-5-ylmethanesulfonylamino)-N-hydroxy-propionamide
(R)-2-(Benzo[b]thiophen-5-ylmethanesulfonylamino)-2-(fluorophenyl)-N-hydroxy-acetamide
(R)-2-(Benzo[b]thiophen-5-ylmethanesulfonylamino)-2-(4-isopropylphenyl)-N-hydroxy-acetamide
(R)-2-(Benzo[b]thiophen-5-ylmethanesulfonylamino)-2-(4-trifluoromethoxyphenyl)-N-hydroxy-acetamide
(R)-2-(Benzo[b]thiophen-5-ylmethanesulfonylamino)-N-hydroxy-2-(4-trifluoromethane-sulfonyloxyphenyl)-acetamide
(R)-2-(Benzo[b]thiophen-5-ylmethanesulfonylamino)-N-hydroxy-3-(phenylmethanesulfonylamino)-propionamide

5. Use of a compound according to any preceding claim for the production of a medicament for the treatment or prophylaxis of disorders in which the overproduction of s-CD23 is implicated.

6. A method for the treatment or prophylaxis of disorders in which the overproduction of s-CD23 is implicated, which method comprises the administration of a compound according to any one of claims 1 to 4 to a human or non-human mammal in need thereof.

7. A pharmaceutical composition for the treatment or prophylaxis of disorders in which the overproduction of s-CD23 is implicated which comprises a compound according to any one of claims 1 to 4 and optionally a pharmaceutically acceptable carrier therefor.

8. Use of a compound according to any one of claims 1 to 4 for the production of a medicament for the treatment or prophylaxis of conditions mediated by TNF.

9. A method for the treatment or prophylaxis of conditions mediated by TNF, which method comprises the administration of a compound according to any one of claims 1 to 4 to a human or non-human mammal in need thereof.

10. A pharmaceutical composition for the treatment or prophylaxis of conditions mediated by TNF, which comprises a compound according to any one of claims 1 to 4 and optionally a pharmaceutically acceptable carrier therefor.

11. A process for preparing a compound according to any one of claims. 1 to 3 which process comprises:
(a) deprotecting a compound of formula (If): 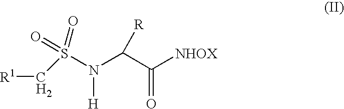
wherein R and R¹ are as defined hereinabove, and X is a protecting group, or
(b) reacting a compound of formula (III): 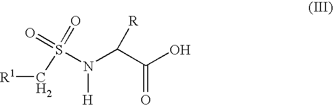
wherein R and R¹ are as defined hereinabove, with hydroxylamine or a salt thereof, or
(c) converting a compound of formula (I) to a different compound of formula (I) as defined hereinabove.

12. A compound of formula (II): 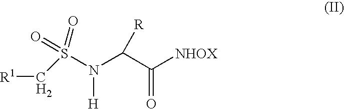
wherein R and R¹ are as defined hereinabove, and X is a protecting group.

13. A compound of formula (m): 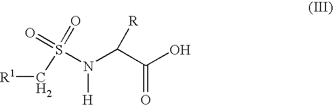
wherein R and R¹ are as defined hereinabove.

14. A process for preparing a compound of formula (VIa): 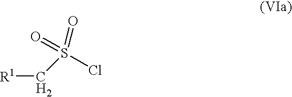
comprising converting a compound of formula (VIII):
R¹—CH₂-Z (VIII)
into a compound of formula (IX): 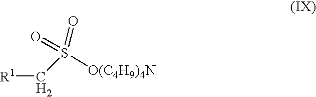
under phase transfer conditions in which the ratio of tetra-n-butylammonium cations to compound of formula (VIII) is greater than 1:1.

15. The process according to claim 14 wherein the ratio of tetra-n-butylammonium cations to compound of formula (VIII) is about 2:1.
